(19) United States
(12) Patent Application Publication
(10) Pub. No.: US 2024/0415976 A1
(43) Pub. Date: Dec. 19, 2024

(54) GPI ANCHORED PROTEINS AND EXTRACELLULAR VESICLES COMPRISING THEM

(71) Applicant: RENEURON LIMITED, Pencoed, Bridgend South Wales (GB)

(72) Inventors: Paul HOLE, Pencoed, Bridgend South Wales (GB); Ruben DEOGRACIAS, Pencoed, Bridgend South Wales (GB); Randolph CORTELING, Pencoed, Bridgend South Wales (GB)

(21) Appl. No.: 18/698,795

(22) PCT Filed: Oct. 10, 2022
(86) PCT No.: PCT/GB2022/052561

§ 371 Date: Apr. 5, 2024

(30) Foreign Application Priority Data
Oct. 8, 2021 (GB) 2114441.5

(51) Int. Cl. A61K 47/69; A61K 35/22; A61K 38/18; C07K 14/475; C07K 14/705

(52) U.S. Cl. CPC A61K 47/6901; A61K 35/22; A61K 38/185; C07K 14/475; C07K 14/70596; C07K 2319/00

(57) ABSTRACT

The present invention provides proteins that are linked to glycosylphosphatidylinositol (GPI). The invention provides a fusion protein comprising a protein of interest and a carboxy-terminal glycosylphosphatidylinositol (GPI) anchor peptide sequence. Proteins of interest include neurotrophins such as BDNF. The invention also provides a fusion protein comprising a protein of interest, a linker peptide and a final carboxy-terminal residue, wherein the final carboxy-terminal residue is covalently linked to glycosylphosphatidylinositol (GPI). The GPI may be anchored to the membrane of an extracellular vesicle. Methods of making and using the fusion proteins and extracellular vesicles of the invention are also provided, including their use in therapy.

### FIELD OF THE INVENTION

This invention relates to a protein of interest comprising glycosylphosphatidylinositol (GPI), and to a fusion protein comprising a GPI anchor sequence. The invention also relates to nucleic acids encoding the proteins of the invention, and to cells expressing the protein of the invention. Extracellular vesicles comprising the protein of the invention embedded in their lipid membrane via GPI are further provided, along with the use of these loaded extracellular vesicles in therapy.

### BACKGROUND OF THE INVENTION

Extracellular vesicles (EVs) are membrane bound vesicles released into the extracellular environment by eukaryotic and prokaryotic cells. Exosomes are a type of EV that originate in the endosomal compartment and are secreted from cells when multivesicular bodies (MVBs) fuse with the plasma membrane. Exosomes typically have a diameter between 30 and 150 nm. Exosomes can contain many types of biomolecule, including proteins, carbohydrates, lipids and nucleic acids. Exosome biogenesis involves the enrichment of their membrane with cholesterol, ceramide and other lipids typically found in detergent-resistant membranes of cells. Proteins are also embedded in the exosome membrane, such as tetraspanins and other transmembrane proteins and receptors that may play a role in their sorting and biological activity (Colombo et al. 2014).

The lumen of exosomes contains proteins and nucleic acids that confer upon the exosomes the capacity to modulate biological effects such as receptor signalling, transcription and translation in target cells. Loading exogenous cargo molecules such as miRNAs and proteins of interest into exosomes has been suggested as a useful way to harness their ability to deliver signals between cells. One method is to introduce biological molecules of interest directly into harvested exosomes. However, despite this being possible at small scales using techniques such as electroporation and lipofection, these techniques have not proven to be repeatable, or reliable at a large scale. Moreover, these techniques may even compromise the structure of the exosome (Janas et al. 2015). WO-A-2021/148022 describes another modification of exosomes, wherein the exosomes are engineered to comprise a targeting moiety with the aim of specifically targeting the exosomes to tissues of interest.

As discussed by Kenari et a/2020 (Methods, Volume 177, pages 103-113, https://doi.org/10.1016/j.ymeth.2020.01.001) EVs have promising therapeutic potential but there are several challenges associated with using EVs before transition from the laboratory to clinical use. Some of these challenges include issues around low yield, isolation and purification methodologies, and efficient engineering (loading) of EVs with therapeutic cargo.

Many neurodegenerative diseases and brain disorders are characterised by gradual and abnormal loss of specific sub-populations of neural cells, including Huntington's Disease, Alzheimer's Disease, Parkinson's Disease, Rett Syndrome and Amyotrophic Lateral Sclerosis. Many ophthalmic conditions similarly involve a loss of functional cells in the eye. Various strategies have been formulated to treat diseases of this class, including the concept of delivering pro-survival signals to the affected neural or eye cells to slow or halt the death of these cells. However, there are as yet few or no approved treatments of this kind, due to poor accessibility of the brain from systemic routes (prevented by the blood-brain barrier) or poor distribution and stability of locally administered agents such as recombinant neuroprotective growth factors.

There remains a need to develop new therapies for treating diseases including neurodegenerative diseases and ophthalmic indications.

### SUMMARY OF THE INVENTION

The invention is based on the development and optimisation of proteins that are linked to glycosylphosphatidylinositol (GPI). Engineering a protein of interest so that it can be linked to GPI during post-translational modification in a cell, targets the protein to extracellular vesicles where the GPI embeds into the lipid membrane. The resulting extracellular vesicles are therefore loaded with the GPI-tagged protein and can be used in a number of applications, including in the treatment of disease.

A first aspect of the invention provides a fusion protein comprising a protein of interest and a carboxy-terminal glycosylphosphatidylinositol (GPI) anchor peptide sequence. The fusion protein may also comprise a linker peptide between the protein of interest and the carboxy-terminal glycosylphosphatidylinositol (GPI) anchor peptide sequence. The linker peptide is typically 2 to 20 amino acid residues in length, for example 3 to 15 amino acid residues or 4 to 10 amino acid residues. In some embodiments, the linker peptide is 4, 5, 6 or 7 amino acids in length. In some embodiments, the linker peptide is 5 amino acids in length. The examples demonstrate that the peptide sequence GAPLE is an effective linker. This may be varied to create a functional variant. A functional variant of this peptide typically includes 1 or 2 substitutions, typically conservative substitutions.

Carboxy-GPI anchor protein sequences are known in the art, as described in more detail below with references to known GPI anchor protein sequences. In one embodiment, the GPI-anchor peptide sequence comprises the GPI anchoring region from human CD59, optionally having the sequence NGGTSLSEKTVLLLVTPFLAAAWSLH (SEQ ID NO: 1) or a functional variant thereof typically having at least 70% identity to that sequence. In another embodiment, the GPI-anchor peptide sequence comprises a GPI anchoring region having at least 80% or at least 90% identity to NGGTSLSEKTVLLLVTPFLAAAWSLH (SEQ ID NO:1).

As will be discussed in greater detail below, processing in the endoplasmic reticulum cleaves most of the GPI anchor sequence and replaces it with GPI covalently bound to the final C-terminal residue of the remaining protein. Typically, all but one residue of the GPI anchor sequence is cleaved and replaced with GPI. Accordingly, all but the amino-terminal residue of the Carboxy-GPI anchor protein sequence is typically cleaved from the protein when the GPI is added. That remaining residue of the anchor sequence then becomes the final C-terminal residue of the protein to which the GPI is attached.

Accordingly, a second aspect of the invention provides a fusion protein comprising a protein of interest, a linker peptide and a final carboxy-terminal residue, wherein the final carboxy-terminal residue is covalently linked to GPI. The final carboxy terminal residue of the construct, is the first (amino-terminal) residue of the carboxy-GPI signal sequence, which remains after cleavage of the other GPI signal sequence residues.

Typically, the final carboxy-terminal residue is asparagine, alanine, aspartic acid, cysteine, glycine, or serine.

In some embodiments of the second aspect, the linker peptide comprises or consists of the sequence GAPLE (SEQ ID NO: 2) peptide or a functional variant thereof.

In certain embodiments, the GPI is embedded in a biological lipid membrane. According to the invention, the biological lipid membrane is typically an extracellular vesicle membrane, for example an exosome membrane.

The protein of interest in all aspects of the invention can, in certain embodiments, be a therapeutic protein, a ligand, an enzyme, a receptor, a growth factor, cytokine, an antibody or an antigen-binding fragment thereof, or a T cell receptor or antigen-binding fragment thereof. The protein of interest may alternatively be a protein useful in a diagnostic procedure, for example a protein that can bind specifically to a marker on a target cell (e.g. an antibody protein of interest binding immunospecifically to its target antigen). When the protein of interest is an antibody, for example a monoclonal antibody or an antigen-binding fragment thereof, the exosomes can be loaded internally or externally with a further molecule or interest, for example a detectable label (e.g. for imaging or diagnosis) or a drug molecule (for therapy) such as a small molecule drug or chemotherapeutic agent. Other drug molecules include proteins, or nucleic acids for example mRNA therapeutics such as mRNA vaccines or RNAi therapeutics such as miRNA or siRNA.

The protein of interest is typically a therapeutic protein, for example an antibody or antigen-binding fragment thereof, a cytokine, growth factor, hormone (e.g. insulin), protein vaccine, enzyme, immunomodulator, interferon, interleukin, thrombolytic protein, clotting factor or TNF superfamily member. An exemplary antibody is anti-Her2 or anti-CD20. Other exemplary antibodies are immune checkpoint inhibitors such as anti-PD1, anti-PD-L1 or anti-CTLA4. In certain embodiments the protein of interest is a neurotrophin, for example Brain-Derived Neurotrophic Factor (BDNF), nerve growth factor (NGF), neurotrophin-3, neuroptrophin-4 or glial cell line-derived neurotrophic factor (GDNF).

In some embodiments, the protein of interest is anchored to the membrane of an extracellular vesicle via GPI and is able to modulate an activity of a target cell. In some embodiments, the target cell is a cell of the central nervous system, for example a neural cell. In some embodiments, modulating activity results in down-regulation of an activity. In some embodiments, modulating activity results in an up-regulation of an activity. In a further embodiment, the protein of the invention results in a reduction of a biological activity, for example receptor signalling or gene expression, by at least 10%, 20%, 30%, 40%, 50%, 60%, 70%, 80%, 90% or greater. Typically, receptor signalling or gene expression is reduced by 60% to 100%. In some embodiments of the invention, the protein of the invention results in an up-regulation of receptor signalling or gene expression. In a further embodiment, the sequence of the invention results in an increase in biological activity, for example gene expression or receptor signalling, by at least 10%, 20%, 30%, 40%, 50%, 60%, 70%, 80%, 90% or greater. Typically, receptor signalling or gene expression is increased by 60% to 100%. Modulation of gene activity can be assessed using western blot, qPCR, fluorescence reporter assay, or luciferase reporter assay. Alternatively, modulation of gene activity can be assessed by measuring the therapeutic outcome, typically by ameliorating the symptoms of the disease of interest.

In some embodiments, the protein of interest is BDNF and the activation of TrkB (Tropomyosin receptor kinase B) in the target cell is increased, typically wherein the increase is at least 10% or at least 20% in TrkB expression compared to a negative control, for example at least 40% compared to a negative control.

In some embodiments, the protein of interest is BDNF and the biological effect is an increase in neurite outgrowth, typically wherein the neurite outgrowth is increased by at least 10% or at least 20% compared to a negative control, for example at least 40% compared to a negative control.

In some embodiments, the protein of interest is BDNF and the biological effect is an increase in retinal ganglion cell (RGC) survival, typically wherein the RGC survival is increased by at least 10% or at least 20% compared to a negative control, for example at least 40% compared to a negative control, typically wherein survival is determined by number of surviving RGC at a given time point for example 72 hours after treatment.

In some embodiments, the protein of interest is BDNF and the biological effect is an increase in retinal ganglion cell (RGC) survival, wherein the RGC survival is increased by at least 50% compared to a negative control wherein survival is determined by number of surviving RGC 72 hours after treatment.

The protein of interest may comprise or consist of a mature sequence. The mature sequence may sit within a longer sequence that comprises a pro-peptide and/or signal sequence. The pro-peptide and/or signal sequence are typically at the amino-terminus of the longer (full length, immature) protein. The pro-peptide and/or signal sequence are typically cleaved to form an active protein. Cleavage typically occurs as a post-translational modification in the endoplasmic reticulum.

The fusion protein of the first or second aspect can be expressed as a monomer. In certain embodiments, two monomers can associate to form a biologically-active dimer, typically a homodimer. This association of two monomers to form a heterodimer or homodimer can occur when two monomers are anchored via GPI in the membrane of an extracellular vesicle such as an exosome. This is particularly useful for generating proteins of interest that are active primarily in dimeric (or multimeric) form. Many growth factors, such as BDNF and PDGF, are active in dimeric form and so can advantageously be GPI-linked and anchored into an extracellular membrane according to the invention. Other dimeric or multimeric proteins include type II restriction enzymes, alcohol dehydrogenase, E. coli alkaline phosphatase, some transcription factors, 14-3-3 proteins, and some clotting factors including Factor Ia (fibrin), Factor XI and Factor XIII.

The fusion protein of the first or second aspect may comprise a peptide that simplifies purification and/or detection, also referred to as a purification tag. An exemplary purification tag comprises or consists of the sequence YPYDVPDYA (SEQ ID NO: 3) or a functional variant thereof. A functional variant may in some embodiments contain 1, 2, 3 or 4 conservative substitutions.

The order of components in the fusion protein of the first aspect is typically N-terminus to C-terminus: protein of interest-optional purification tag-linker peptide-GPI anchor protein sequence.

The order of components in the fusion protein of the second aspect is typically N-terminus to C-terminus: protein of interest-optional purification tag-linker peptide-GPI-optional EV membrane.

In one embodiment, the protein of interest comprises or consists of mature BDNF, the optional purification tag comprises or consists of YPYDVPDYA, the linker peptide comprises or consists of GAPLE, and the GPI-anchor protein comprises or consists of NGGTSLSEKTVLLLVTPFLAAAWSLH.

In one embodiment, a fusion protein of the first aspect invention comprises full-length BDNF, a GAPLE linker and the GPI anchor sequence from CD-59. An exemplary amino acid sequence in this embodiment is:

(SEQ ID NO: 4)
MTILFLTMVISYFGCMKAAPMKEANIRGQGGLAYPGVRTHGTLESVNGPK
AGSRGLTSLADTFEHVIEELLDEDQKVRPNEENNKDADLYTSRVMLSSQV
PLEPPLLFLLEEYKNYLDAANMSMRVRRHSDPARRGELSVCDSISEWVTA
ADKKTAVDMSGGTVTVLEKVPVSKGQLKQYFYETKCNPMGYTKEGCRGID
KRHWNSQCRTTQSYVRALTMDSKKRIGWRFIRIDTSCVCTLTIKGAPLEN
GGTSLSEKTVLLLVTPFLAAAWSLH.

A purification tag may optionally be included between the BDNF (plain text) and GAPLE (underlined) sequences. An example of a purification tag is YPYDVPDYA.

In one embodiment, a fusion protein of the first aspect of the invention comprises or consists of a protein having at least 80% or at least 90% identity to the sequence

(SEQ ID NO: 4)
MTILFLTMVISYFGCMKAAPMKEANIRGQGGLAYPGVRTHGTLESVNGPK
AGSRGLTSLADTFEHVIEELLDEDQKVRPNEENNKDADLYTSRVMLSSQV
PLEPPLLFLLEEYKNYLDAANMSMRVRRHSDPARRGELSVCDSISEWVTA
ADKKTAVDMSGGTVTVLEKVPVSKGQLKQYFYETKCNPMGYTKEGCRGID
KRHWNSQCRTTQSYVRALTMDSKKRIGWRFIRIDTSCVCTLTIKGAPLEN
GGTSLSEKTVLLLVTPFLAAAWSLH.

In one embodiment of the second aspect, a fusion protein of the invention comprises mature BDNF, a GAPLE linker and the N-terminal residue (only) of the GPI anchor sequence from CD-59. An exemplary amino acid sequence in this embodiment is:

(SEQ ID NO: 5)
HSDPARRGELSVCDSISEWVTAADKKTAVDMSGGTVTVLEKVPVSKGQLK
QYFYETKCNPMGYTKEGCRGIDKRHWNSQCRTTQSYVRALTMDSKKRIGW
RFIRIDTSCVCTLTIKGAPLEN

In this embodiment, the C-terminal asparagine residue is typically covalently linked to glycosylphosphatidylinositol.

A purification tag may optionally be included between the BDNF (plain text) and GAPLE (underlined) sequences. An example of a purification tag is YPYDVPDYA.

In one embodiment, a fusion protein of the second aspect of the invention comprises or consists of a protein having at least 80% or at least 90% identity to the sequence

(SEQ ID NO: 5)
HSDPARRGELSVCDSISEWVTAADKKTAVDMSGGTVTVLEKVPVSKGQLK
QYFYETKCNPMGYTKEGCRGIDKRHWNSQCRTTQSYVRALTMDSKKRIGW
RFIRIDTSCVCTLTIKGAPLEN.

The term fusion protein includes recombinant fusion proteins made by expressing polypeptide sequences, typically in a cell, from a recombined nucleic acid sequence. The invention typically uses recombinant nucleic acid technology. Nonetheless, it is also possible to create fusion proteins synthetically, for example by chemical conjugation, combinatorial chemistry or by using an isopeptide bond to link two proteins covalently for example using the SpyTag/SpyCatcher system described by Zakeri et al 2012 (PNAS Mar. 20, 2012 109 (12) E690-E697).

A third aspect of the invention provides a polynucleotide encoding a fusion protein according to the first aspect or the second aspect.

A fourth aspect of the invention provides a plasmid, virus or vector comprising the polynucleotide of the third aspect. In some embodiments, the polynucleotide encoding a fusion protein is under the control of a promoter sequence, optionally an inducible promoter.

In one embodiment, a polynucleotide expression cassette comprises: a ubiquitous or inducible promoter region; a cell specific promoter or a promoter with specific and/or selected transcription factor binding sites; an enhancer or repressor sequence in the 5′ upstream sequence of the fusion protein; and/or a 3′ enhancer, repressor or stabilizing sequence in the 3′ downstream sequence of the fusion protein.

In some embodiments, the polynucleotide vector of the invention comprises an adenoviral vector, adeno-associated viral vector, the pEF1-alpha, pTK, pCAG, pSV and the pCMV series of plasmid vectors, vaccinia, and retroviral vectors, or baculovirus. In a particular embodiment, the vector of the invention comprises a lentiviral vector plasmid. In some embodiments the vector comprises the promoters for cytomegalovirus (CMV), CAG, EF1-alpha, TK and SV40.

A fifth aspect of the invention provides an extracellular vesicle comprising a fusion protein according to the second aspect, wherein the protein is covalently attached to GPI and the GPI is anchored in the extracellular vesicle membrane. This aspect of the invention therefore provides an extracellular vesicle loaded with the protein of interest.

The fusion protein may, in some embodiments, be present as a homodimer in the extracellular vesicle membrane. The extracellular vesicle is typically an exosome. The extracellular vesicle may, in addition to the GPI-anchored protein, comprise an additional exogenous protein, nucleic acid, lipid, drug, prodrug, therapeutic agent or diagnostic agent. The extracellular vesicle, e.g. exosome, may be from a stem cell, optionally a neural stem cell. The stem cell may optionally be conditionally-immortalised, for example using the c-MycER gene. An example of a conditionally-immortalised neural stem cell is the CTX0E03 cell line.

A sixth aspect of the invention provides a host cell comprising a fusion protein, a polynucleotide, a plasmid, virus or vector, or an extracellular vesicle according to any of the preceding aspects. The cell that can be used is not particularly limited, and the skilled person will be aware that a wide range of cells can be used. In some embodiments, the cell is a HEK293 or HEK293T cell, as is well-known in the art for expressing proteins and producing recombinant retroviruses. In some embodiments the host cell is conditionally-immortalised, for example using the c-MycER gene. In some embodiments, the host cell is a stem cell, optionally a neural stem cell or a mesenchymal cell. In a particular embodiment the cell is a neural stem cell. In a further embodiment, the cell is a CTX0E03 cell (deposited by the applicant of this patent application at the ECACC with Accession No. 04091601). In another embodiment, the cell is optionally a partially differentiated stem cell. In further embodiments, the cell can be a pluripotent stem cell, for example an induced pluripotent stem cell (iPSC). The cell can also be a cell that is differentiated from an iPSC, which may be a stem cell derived from an iPSC or a terminally-differentiated cell derived from an iPSC. Exemplary iPSCs and their progeny are derived from CTX0E03 cells, as described in WO-A-2020074925 and PCT/GB2021/050905 (both incorporated herein by reference in their entireties). The vector or other coding nucleic acid can be introduced into the cell by any known method of introducing a vector or nucleic acid into the cell. Such methods may include but are not limited to transfection using a cationic lipid reagent, electroporation, and viral transduction.

In some embodiments, extracellular vesicles comprising the GPI-anchored protein of interest are collected from the host cell of the sixth aspect. Typically, the EV's are harvested from the conditioned culture media in which the cells are bathed during culture.

A seventh aspect of the invention provides a pharmaceutical composition comprising a fusion protein, a polynucleotide, a plasmid, virus or vector, an extracellular vesicle, or a host cell, according to any of the preceding aspects, and a pharmaceutically acceptable carrier, diluent or excipient. In some embodiments, the pharmaceutical composition comprises an exosome loaded with a GPI-tethered protein of the invention.

An eighth aspect of the invention provides a pharmaceutical composition comprising a fusion protein, a polynucleotide, a plasmid, virus or vector, an extracellular vesicle, or a host cell, according to any of the preceding aspects, for use in a method of treatment or a method of diagnosis. In certain embodiments, the treatment is the treatment or prevention of a central nervous system disease or disorder, optionally Huntington's Disease, Alzheimer's Disease, Multiple Sclerosis, Parkinson's Disease, Rett Syndrome or Amyotrophic Lateral Sclerosis. In some embodiments, an exosome loaded with a GPI-tethered protein of the invention is provided for these therapeutic uses, for example when the protein of interest is a neuroprotective protein such as BDNF.

In other embodiments the treatment is of an ophthalmic indication. The ophthalmic condition may involve the loss of functional cells such as retinal cells. In some embodiments, the ophthalmic indication is retinitis pigmentosa or glaucoma. Other diseases include wet macular degeneration, dry macular degeneration, dry eye disease, diseases of the retina or diseases of the cornea. In some embodiments, an exosome loaded with a GPI-tethered protein of the invention is provided for these therapeutic uses, for example when the protein of interest is a neuroprotective protein such as BDNF.

A ninth aspect of the invention provides a method of expressing a fusion protein according to the first or second aspect, or of producing an extracellular vesicle according to the fifth aspect, wherein the method comprises expressing the fusion protein in a host cell, optionally from a recombinant expression cassette or expression vector. The host cell, in some embodiments, may be from a conditionally-immortalised cell line. An example of a suitable cell line is the CTX0E03 cell line. Some embodiments of the ninth aspect comprise the further step of harvesting or collecting extracellular vesicles, for example exosomes, comprising the fusion protein from the host cell or host cell media.

In some embodiments, the method of expressing a fusion protein of the ninth aspect comprises:
cleavage of an N-terminal signal sequence and/or pro-peptide sequence present on the protein of interest; and/or
cleavage of C-terminal residues of the GPI anchor protein sequence and attachment of GPI to the remaining residues of the GPI anchor protein sequence; and/or embedding the fusion protein into the exosome membrane via GPI attached to the GPI anchor sequence.

A cleavage step may, in one embodiment, convert a pro-sequence to a mature sequence by removing all or part of the pro-sequence. This occurs, for example, to convert pro-BDNF to mature BDNF.

One or more of the cleavage steps can be carried out co-translationally or post-translationally, by endogenous enzymes in the host cell.

A further aspect provides a method of loading exosomes with a protein of interest comprising producing exosomes from a cell comprising a nucleotide construct of the invention. In particular embodiments, the invention enables the loading of any protein of interest into the membrane of an extracellular vesicle such as an exosome.

In a further aspect of the invention, a method of preparing extracellular vesicles (e.g. exosomes) comprising the pre-GPI-tagged protein of the invention is provided.

The method of preparing exosomes comprising the GPI-tagged protein typically comprises culturing cells and harvesting conditioned media. In an embodiment, the method further comprises the optional steps of purification of the extracellular vesicles and validation of the extracellular vesicles. In a further embodiment, the method comprises culturing the cells, harvesting of conditioned media, and optionally purification, and optionally validation of the exosomes. The method may also comprise packaging or formulating the exosomes into a pharmaceutical product or unit dose. In some embodiments, an exosome that is obtained or obtainable from the method is provided. As will be understood, exosomes or other extracellular vesicles may be purified by any method known in the art.

A further aspect of the invention provides a method of delivering a protein of interest to a target cell, using an exosome loaded with a GPI-anchored protein of interest produced according to the invention. This delivery can be in vitro or in vivo. In some embodiments, the target cell is a cell of the central nervous system, for example a neural cell. In an embodiment, the method comprises contacting the target cell with the loaded exosomes. In a further embodiment, the method optionally comprises first, a step comprising isolating the exosomes loaded protein, and second, a step comprising contacting the target cell with the loaded exosomes. In some embodiments, the contacting the target cell with the loaded exosomes step is for at least 1 hour, 2 hours, 3 hours, 4 hours, 5 hours, 24 hours, or greater. In another embodiment the contacting step occurs at 37° C.

A further aspect of the invention provides a method of modulating gene activity in a target cell, comprising administering an exosome comprising a biologically active protein of interest, generated according to the invention. In some embodiments, the target cell is a cell of the central nervous system, for example a neural cell. In some embodiments, modulating gene activity results in down-regulation of gene expression. In some embodiments, modulating gene activity results in an up-regulation of gene expression. In some embodiments of the invention, the exogenous nucleotide sequence of the invention results in a down-regulation of gene expression. In a further embodiment, the exogenous nucleotide sequence of the invention results in a reduction of gene expression by at least 10%, 20%, 30%, 40%, 50%, 60%, 70%, 80%, 90% or greater. Typically, gene expression is reduced by 60% to 100%. In some embodiments of the invention, the exogenous nucleotide sequence of the invention results in an up-regulation of gene expression. In a further embodiment, the exogenous nucleotide sequence of the invention results in an increase in gene expression by at least 10%, 20%, 30%, 40%, 50%, 60%, 70%, 80%, 90% or greater. Typically, gene expression is increased by 60% to 100%. Modulation of gene activity can be assessed using western blot, qPCR, fluorescence reporter assay, or luciferase reporter assay. Alternatively, modulation of gene activity can be assessed by measuring the therapeutic outcome, typically by ameliorating the symptoms of the disease of interest.

### BRIEF DESCRIPTION OF THE DRAWINGS

FIG. 1: Schematic of the ExoBDNF construct and predicted localisation on the exosomes

FIG. 2: ExoBDNF exosomes bind to antigen-binding domain of TrkB

FIG. 3: BDNF is detected in ExoBDNF preparations by Western Blot

FIG. 4: ExoBDNF drives neurite outgrowth in an in vitro dopaminergic neuron model

FIG. 5: ExoBDNF drives transcription of the BDNF target gene, c-fos, in murine brains in vivo

FIG. 6: Schematic of GPI addition and transport

FIG. 7: Schematic of the BNDF fusion protein processing and GPI conjugation in vivo

FIG. 8: Exemplary GPI anchor sequences that can be used according to the invention.

FIG. 9: Evaluation of BDNF-specific activity in ExoBDNF using a TrkB activity reporter assay.

FIG. 10: Comparison of neurite growth induction in retinal ganglion cells.

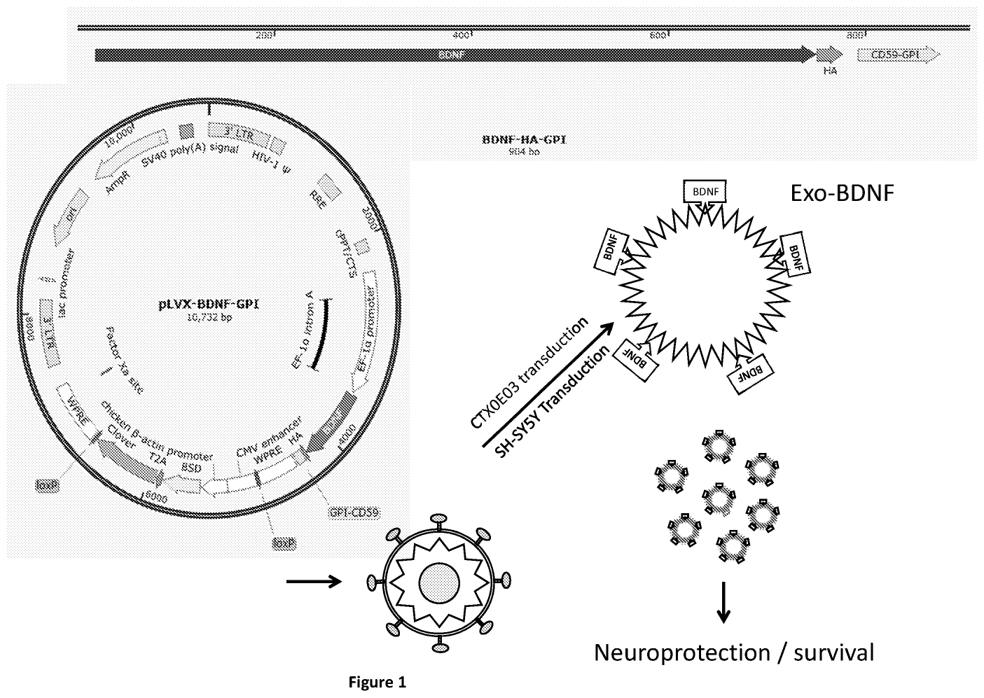

### DETAILED DESCRIPTION OF THE INVENTION

The present inventors have investigated how a protein of interest can be effectively loaded onto extracellular vesicles (EVs) using glycosylphosphatidylinositol (GPI) to anchor the protein of interest into the membrane of the EV. This is achieved by linking the protein of interest to a polypeptide sequence that mediates GPI attachment to the protein. This polypeptide sequence is referred to as a GPI anchor or GPI anchor sequence. The linkage between the protein of interest and GPI anchor is typically provided by expressing the protein of interest and GPI anchor as a recombinant fusion protein, although other techniques to link two or more polypeptide sequences of interest are known and can be used.

The Examples demonstrate the successful loading of brain derived neurotrophic factor (BDNF) into exosomes using a GPI anchor based on the GPI anchor sequence from CD59 and with a short peptide linker separating the BDNF and GPI anchor. This system can be applied to other proteins of interest, other extracellular vesicles, other GPI anchor sequences and other linkers. The exemplified system is introduced into host cells using a retrovirus and the expressed protein is then presented on the surface of exosomes produced by the host cell. Other expression systems can be used, as will be apparent to the skilled person.

The exemplified system loads the protein into exosomes from neural stem cells. Other cell types can be used, as will be apparent to the skilled person.

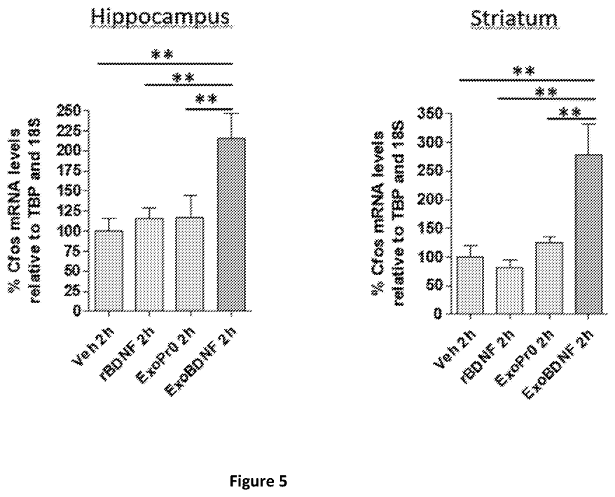

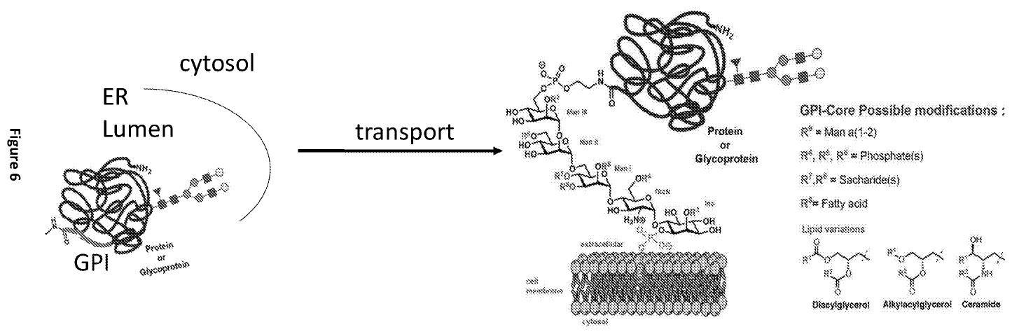

In the exemplified system, BDNF activity in vivo is surprisingly shown following intrathecal injection of the BDNF-GPI loaded exosomes in animals, with consequent evidence of up-regulation of target gene expression in the brain, specifically in the hippocampus and the striatum. This is particularly surprising when it is noted that recombinant BDNF administered in “naked” form (not embedded in a vesicle membrane) was observed to be ineffective in this same experiment, as can be seen in FIG. 5. These findings therefore indicate the therapeutic potential of these BDNF-modified exosomes and indicate the suitability of this GPI-based system to load other proteins of interest onto exosomes, in particular therapeutic proteins.

The system of the invention is also particularly suitable for use with proteins of interest that are active upon dimerization. Without being bound by theory, the use of the GPI anchor and linker system described herein may provide enhanced stability and activity of dimeric proteins when anchored in the EV membrane.

The exemplified system is particularly suited to deliver exosomes loaded with therapeutic proteins to the central nervous system, in particular to the brain for example the hippocampus and/or striatum. This CNS, e.g. brain, targeting can be provided when the exosomes are administered peripherally such as by intravenous or intraperitoneal administration, or is administered intrathecally.

The invention provides methods for the modification of any host cell to generate EVs, typically exosomes, that are loaded with any protein of interest. A purified exosome of the invention can then be used systemically or locally in vivo for use in therapy. The exosome containing the protein sequence of interest can offer improved biodistribution, systemic stability, and improved target tissue uptake compared to naked proteins.

### Protein of Interest

Any protein of interest can be used according to the invention. Typically, the protein will have therapeutic or diagnostic application, such as an active biological molecule or an agent with specific affinity for a target such as an antibody or antigen-binding fragment thereof. For diagnostic use, the protein may be detectable itself or may comprise a separate detectable label.

The protein of interest may in some embodiments be a diagnostic protein, for example a protein that can bind specifically to a marker on a target cell (e.g. an antibody protein of interest binding immunospecifically to its target antigen).

When the protein of interest is an antibody, for example a monoclonal antibody or an antigen-being fragment thereof, the exosomes can be loaded internally or externally with a further molecule or interest, for example a detectable label (e.g. for imaging or diagnosis) or a drug molecule (for therapy) such as a small molecule drug or chemotherapeutic agent.

The protein of interest can, in certain embodiments, be a therapeutic protein, a ligand, a receptor, a growth factor, cytokine, an antibody or an antigen-binding fragment thereof, or a T cell receptor or antigen-binding fragment thereof.

Therapeutic proteins of interest are particularly provided, for example an antibody or antigen-binding fragment thereof, a cytokine, growth factor, hormone (e.g. insulin), protein vaccine, enzyme, immunomodulator, interferon, interleukin, thrombolytic protein, clotting factor or TNF superfamily member. In certain embodiments the protein of interest is a neurotrophin, for example Brain-Derived Neurotrophic Factor (BDNF), nerve growth factor (NGF), neurotrophin-3 or neuroptrophin-4.

In certain embodiments, the therapeutic protein is a neuroprotective growth factor, optionally Brain-Derived Neurotrophic Factor (BDNF), ciliary neurotrophic factor (CNTF), glial cell-line-derived neuroptrophic factor (GDNF) or nerve growth factor (NGF).

In one embodiment, the protein of interest is brain derived neurotrophic factor (BDNF). Similar to all neurotrophins, human BDNF is initially synthesized as a precursor form (pro-BDNF), consisting of a prodomain of 129 amino acids and a mature domain of 118 amino acids. The mature domain forms a cysteine knot structure, leading to non-covalent dimerization of the mature domains. BDNF cDNA encodes a 247 amino acid residue precursor protein with a signal peptide and a proprotein that are cleaved to yield the 119 amino acid residue mature BDNF (His129 to Arg247). The amino acid sequence of mature BDNF is identical in all mammals examined.

Human BDNF has UniProt accession number P23560, pasted below with the mature sequence underlined:

```
(SEQ ID NO: 6)
                10 20 30 40
MTILFLTMVI SYFGCMKAAP MKEANIRGQG GLAYPGVRTH
                50 60 70 80
GTLESVNGPK AGSRGLTSLA DTFEHVIEEL LDEDQKVRPN
                90 100 110 120
EENNKDADLY TSRVMLSSQV PLEPPLLELL EEYKNYLDAA
              130 140 150 160
NMSMRVRRHS DPARRGELSV CDSISEWVTA ADKKTAVDMS
              170 180 190 200
GGTVTVLEKV PVSKGQLKQY FYETKCNPMG YTKEGCRGID
              210 220 230 240
KRHWNSQCRT TQSYVRALTM DSKKRIGWRF IRIDTSCVCT
LTIKRGR
```

Functional variants of full length BDNF or mature BDNF can be used according to the invention. Functional variants retain at last 50%, typically at least 70% and more typically about 100% of the function of the native BDNF shown above. The assays used in the Examples can also be used to compare the activity of a functional variant of BDNF to native BDNF.

Optionally, the final three amino-terminal residues (“RGR”) of the BDNF sequence shown above are not present in the full-length or mature BDNF used according to the invention. Functional variants of this alternative BDNF sequence are also provided according to the invention, for example that are at least 70%, at least 80% or at least 95% identical to the alternative BDNF sequence (lacking the amino terminal RGR). Without wishing to be bound by theory, the inventors observed (data not shown) that exclusion of the C-terminal “RGR” amino acids can promote stability of BDNF fusion proteins where proteins are fused to the C-terminus of BDNF, and improve the yield of the expressed fusion protein compared to the complete sequence including these three residues. Accordingly, in some embodiments a BDNF fusion protein of the invention can be optimised by removing the final RGR residues from the expressed protein sequence, e.g by providing a BDNF nucleic acid coding sequence that does not encode these residues. This is expected to optimise the anchoring of the BDNF onto extracellular vesicles via GPI.

One exemplary assay for BDNF activity is a cell proliferation assay using BaF mouse pro-B cells transfected with TrkB, where the expected ED50 is 0.2-2 ng/ml. The specific activity of recombinant Human BDNF is approximately 1.3×10³ units/μg, which is calibrated against recombinant human BDNF WHO Standard (NIBSC code: 96/534).

A functional variant of BDNF can also be measured by its binding ability in a functional ELISA. When Recombinant Human TrkB Fc Chimera (R&D Systems Catalog #688-TK) is coated at 1 μg/mL, Recombinant Human BDNF binds with an apparent Kd<1 nM.

The sequence of a functional variant of full length BDNF is typically at least 70%, at least 80% or at least 90%, for example at least 95% identical to the amino acid sequence of full length native human BDNF.

The sequence of a functional variant of mature BDNF is typically at least 70%, at least 80% or at least 90%, for example at least 95% identical to the amino acid sequence of mature native human BDNF.

BDNF is known in the art and acts as a signal for proper axonal growth and synapse formation. It is a strong neuroprotectant in the CNS and is essential for learning and memory. Decreased BDNF is associated with CNS pathologies and neuropsychiatric disorders, including Huntington's disease, Alzheimer's disease, Multiple Sclerosis, Parkinson's disease, depression, epilepsy, Rett Syndrome (ASD-related) and ischemic stroke. BDNF that is active in vivo, as provided by the exosomes comprising BDNF according to the invention, can therefore provide a therapeutic agent for these conditions.

BDNF needs to be dimerised in order to activate its receptor Trk-B, and BDNF cannot be used effectively as a therapeutic agent in its natural form, where it has been observed to be poorly distributed and unstable in vivo.

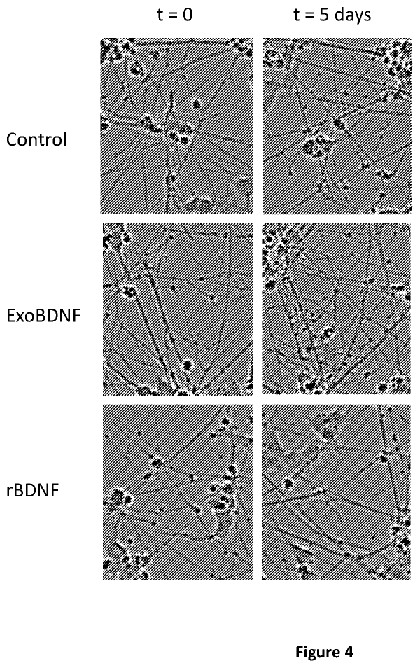

However, surprisingly, the Examples (e.g. FIGS. 4 and 5) show that BDNF anchored via GPI on an exosome according to the invention stabilises and increases the potency of BDNF, compared to native dimeric BDNF. Accordingly, the invention turning an unusable agent into a potent agent with therapeutic application. FIGS. 5, 9 and 10 confirm that the GPI-anchored BDNF exosomes activates downstream gene expression, can activate the TrkB receptor and are biologically active (by increasing the biological effect of neurite outgrowth).

The fusion construct of the invention typically expresses BDNF as a monomer fused to a GPI anchor sequence. The GPI anchor sequence is cleaved after translation and replaced with the GPI by the action of a transamidase enzyme in the cell (as summarised, for example, in FIG. 6). Without being bound by theory, it would appear that the fusion protein of the invention allows BDNF dimerisation to take place, between two separate BDNF-GPI fusion proteins in the membrane of the extracellular vesicle. Again without being bound by theory, the exemplified exosomal GPI-anchored BDNF may be improving distribution and stability by providing a less appealing target for degradation by proteases, and/or or the properties of the exosome may lead to more favourable and prolonged tissue distribution than the protein alone. A further explanation for the increased potency could be the way that the BDNF is presented to the receptor is directly responsible for the increased potency, either by presenting in a novel orientation/configuration with higher affinity for the receptor, or by multivalent activation of Trk-B.

### GPI Anchor Sequence

The cellular process for the attachment of GPI to proteins in vivo is well known and well-characterised, as described for example by: Gerber et al JBC 1992 Vol. 267 No. 17 pages 12168-73; Kinoshita 2020 Biosynthesis and biology of mammalian GPI-anchored proteins. Open Biol. 10:190290 doi.org/10.1098/rsob.190290; and Chapter 12 of Essentials of Glycobiology [Internet]. 3rd edition, Varki A, Cummings R D, Esko J D, et al., editors. Cold Spring Harbor (NY): Cold Spring Harbor Laboratory Press, all of which are incorporated by reference in their entireties. The biomedical applications of GPI-anchored proteins are also known in the art, as discussed by Medof et al (FASEB J. 10, 574-586, 1996), US-A-2016/0271234 and Nelson & Muenchmeier (Oncolmmunology 2013; 2: e26619) and as reviewed by Heider et al J. Lipid Res. 2016, 57:1778-1788. It is also known that some EVs are enriched in lipid raft-associated lipids, including sphingolipids and cholesterol, and proteins, including glycosylphosphatidylinositol (GPI)-anchored proteins (de Gassart et al, Blood Volume 102, Issue 13, 15 Dec. 2003, Pages 4336-4344).

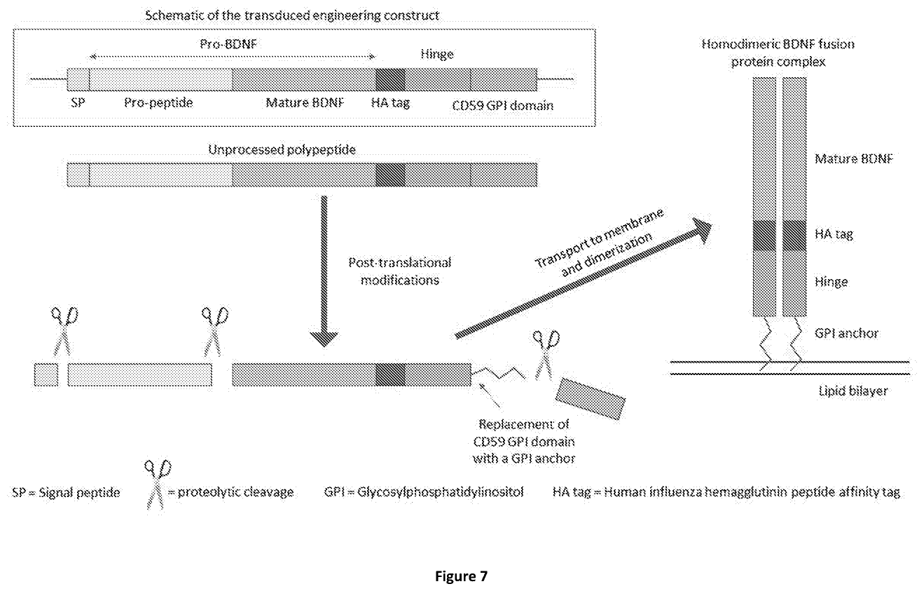

As is known in the art and as summarised in FIGS. 6 and 7, GPI-anchoring is a post-translational modification. The GPI is assembled on the endoplasmic reticulum (ER) membrane and is transferred to the target protein immediately after its translocation into the ER lumen, with concomitant cleavage of a carboxy-terminal GPI-anchor sequence by a transamidase enzyme. Lipid remodeling and/or carbohydrate side-chain modifications may then occur in the ER and after transport to the Golgi apparatus.

C-terminal GPI anchor sequences are known in the art. Hundreds of GPI-anchored proteins have been identified in many eukaryotes, ranging from protozoa and fungi to humans. For example, Table 1 of Gerber et al (ibid.) describes a number of known carboy-terminal GPI sequences, all of which could be used according to the present disclosure. Table 1 of Gerber et al and each GPI anchor sequence in it is expressly incorporated by reference.

Any suitable GPI anchor sequence may be used according to the invention. Examples of mammalian proteins having known C-terminal GPI anchor sequences include CD59, CD52, CD55 (DAF), CD87 (uPAR), Neural cell adhesion molecule 120 (NCAM-120), Neural cell adhesion molecule TAG-1, CD58, FcγIII receptor, Ciliary neurotrophic factor receptor (CNTFR) a subunit, Glial-cell-derived neurotrophic factor receptor (GDNFR) a subunit, CD14, and the Glypican family of GPI-anchored proteoglycans.

The carboxy-terminal GPI-anchor peptide has three domains: (1) three relatively small amino acids (Ala, Asn, Asp, Cys, Gly, or Ser) located at ω, ω+1, and ω+2, where ω is the amino acid attached to the GPI anchor and where ω+1 and ω+2 are the first two residues of the cleaved peptide; (2) a relatively polar domain of typically five to 10 residues; and (3) a hydrophobic domain of typically 15-20 hydrophobic amino acids. These anchor sequences are easily identified by eye and by automated algorithms.

The Examples demonstrate the successful use of the GPI anchor sequence from CD59, having the sequence NGGTSLSEKTVLLLVTPFLAAAWSLH (SEQ ID NO: 1). In this sequence, the first three residues NGG are the ω, ω+1, and ω+2 residues, respectively. Therefore, upon addition of the GPI and cleavage of the signal peptide in the ER, only the w residue “N” is retained in the protein, and it is that residue to which the GPI is covalently linked. The remainder of the GPI anchor protein (i.e. GGTSLSEKTVLLLVTPFLAAAWSLH, SEQ ID NO: 7) is no longer part of the protein once the GPI is attached.

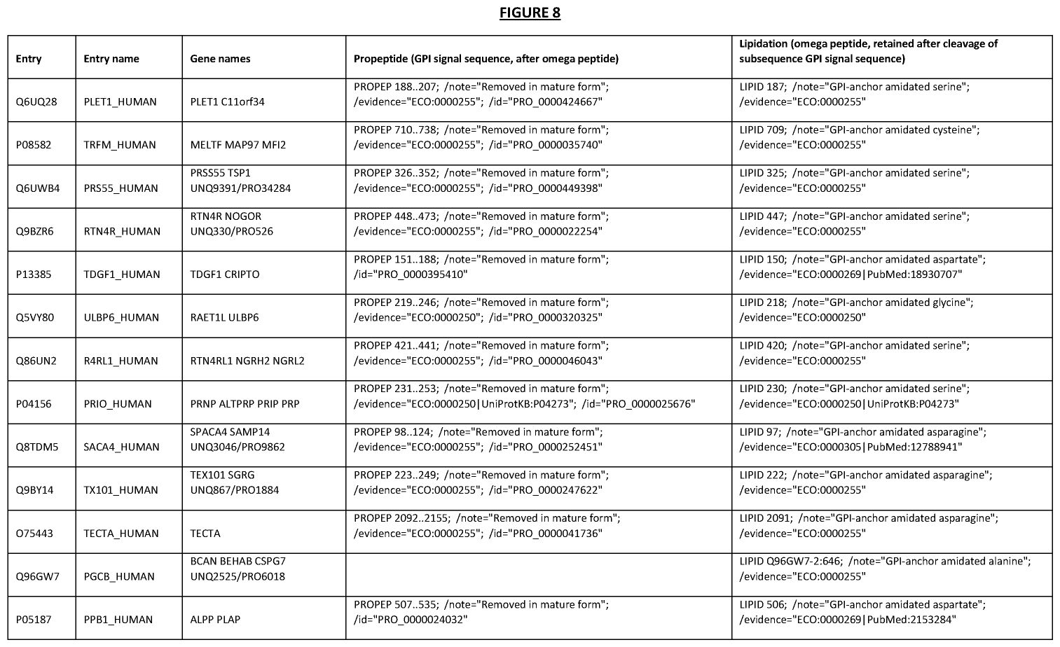

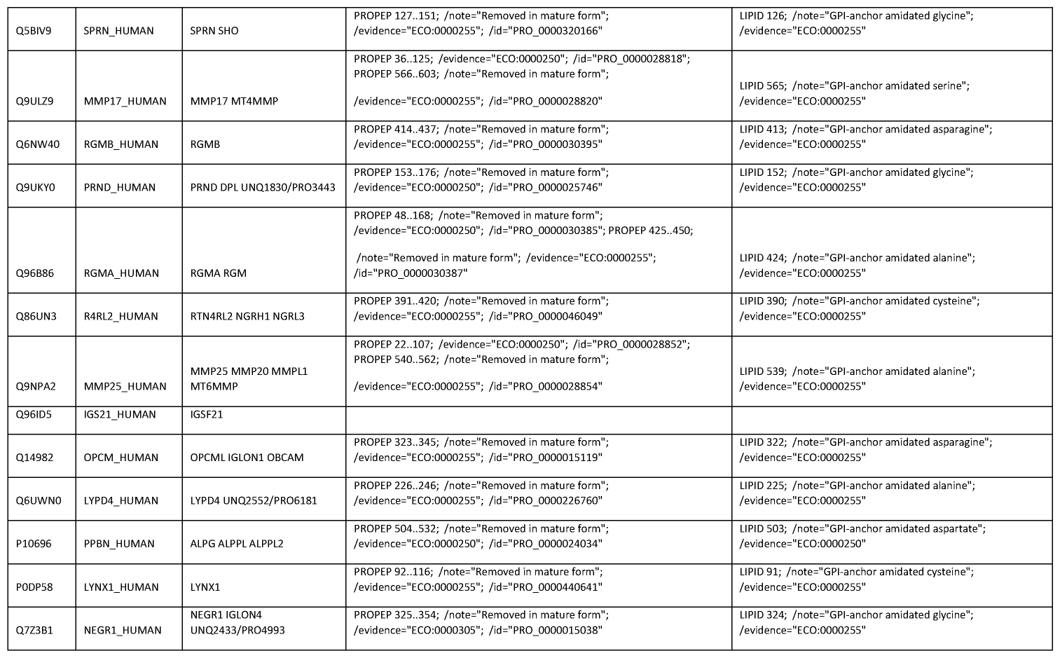

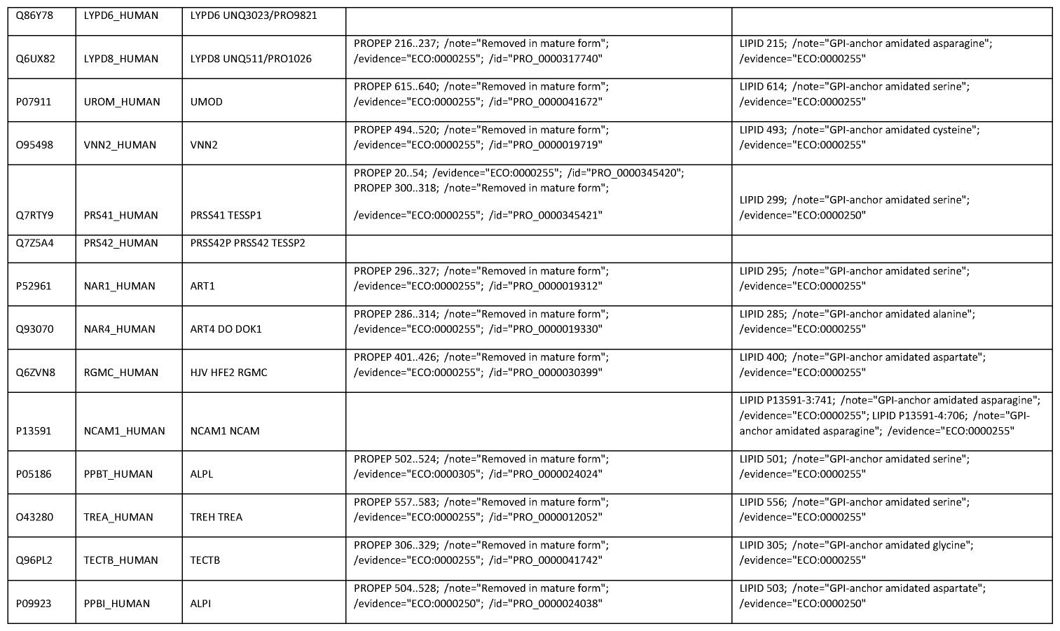

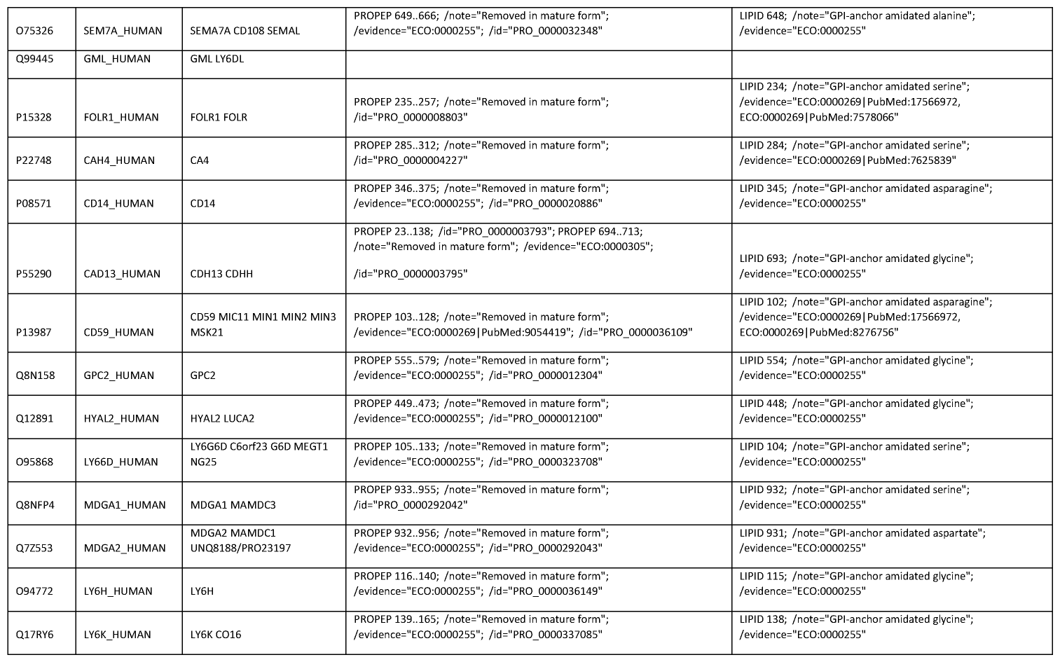

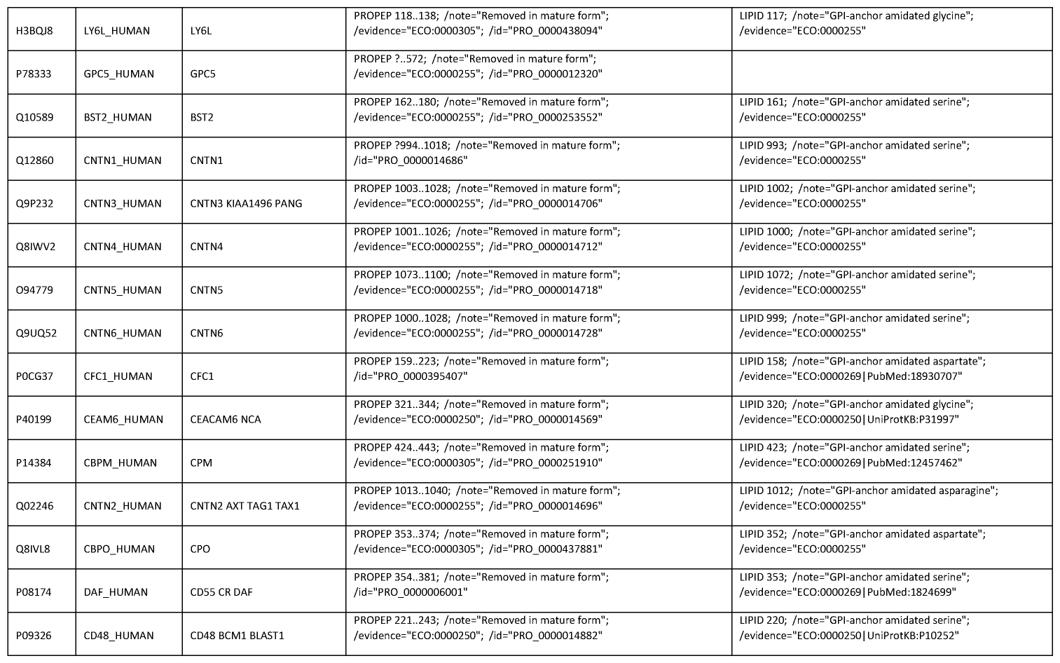

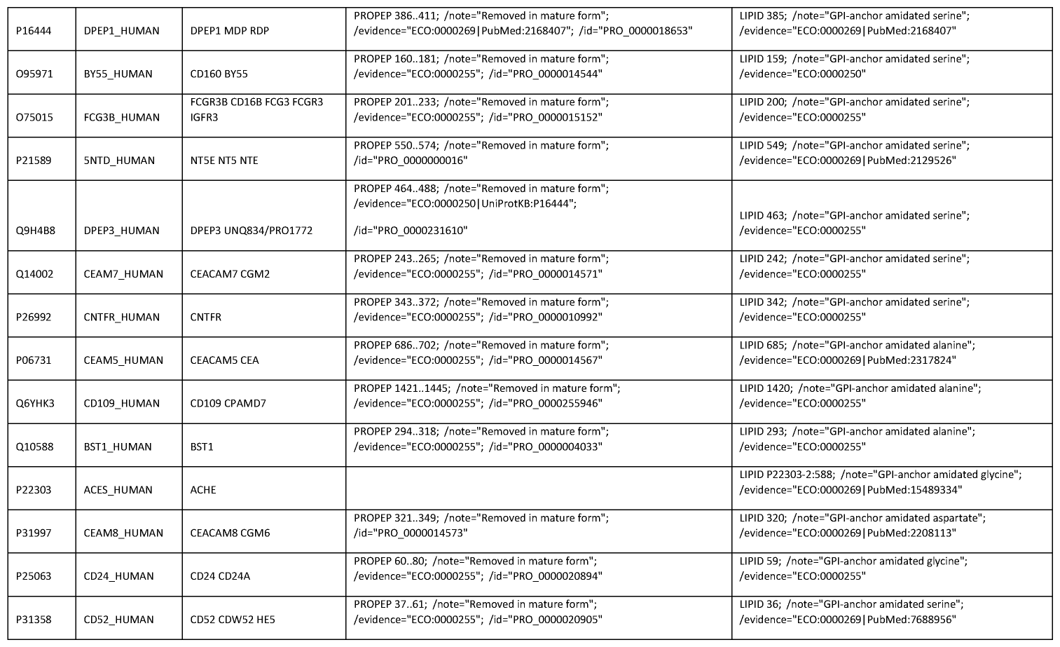

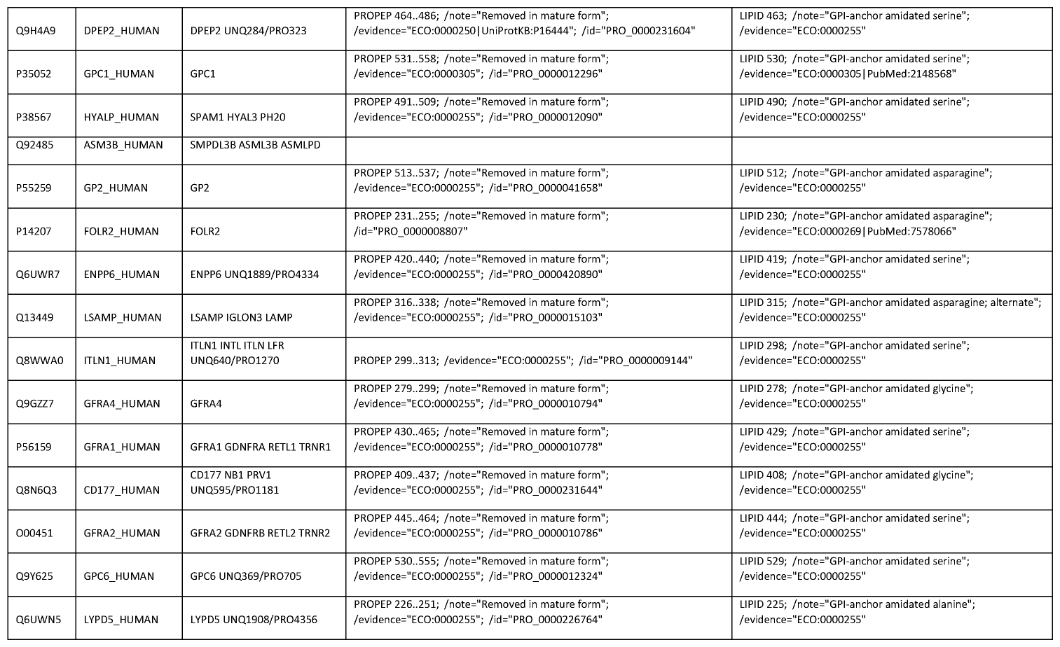

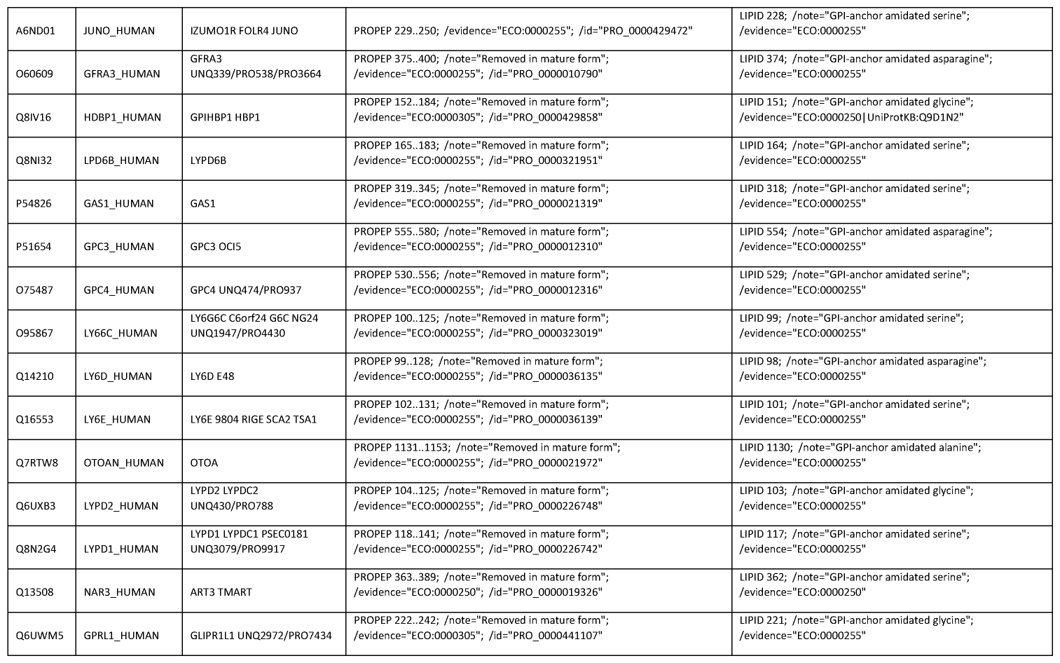

Other non-limiting examples of suitable GPI anchor sequences that can be used according to the invention are also described herein. A detailed disclosure of human proteins that contain GPI anchor sequences is provided in FIG. 8. In this table, each protein is identified in the first column by its UNIPROT accession number. The residue numbers of the cleaved GPI anchor sequence (ω+1 onwards) in each protein is identified in the fourth column, while the ω residue that becomes attached to GPI is identified in the fifth column. For example, for the first entry the protein with UNIPROT accession number Q6UQ28 (Human Plet1) has a GPI anchor sequence at residues 187 to 207. This is indicated by the notation in column 4 that the “PROPEP 188 . . . 207” sequence is cleaved (i.e. residue 188 is ω+1) and the “LIPID 187” notation in column 5 indicating that amino acid 187 is the w residue linked to the GPI. The complete GPI sequence present in each protein in this table is therefore provided by the combination of residues identified in column 4 and column 5. Where there are multiple peptides mentioned in column 4, the cleavable GPI anchor sequence (ω+1 onwards) is the most C-terminal sequence described, i.e. with the highest residue numbering.

Each of the GPI anchor sequences in FIG. 8 can be used according to the invention and each is explicitly provided as an embodiment of the invention. Functional variants of each of these GPI sequences are also provided as embodiments of the invention.

Examples of GPI anchor sequences that can be used according to the invention are also described below:

CD52
(SEQ ID NO: 8)
(36)SASSNISGGIFLFFVANAIIHLFCFS(61)

37-61 cleaved to form GPI anchor, 36 (S underlined) is the AA binding the GPI anchor itself (ω)

CD55 (DAF)
(SEQ ID NO: 9)
(353)SGTTRLLSGHTCFTLTGLLGTLVTMGLLT (381)

354-381 cleaved to form GPI anchor, 353 (S underlined) is the AA binding the GPI anchor itself (ω)

CD87 (uPAR)
(SEQ ID NO: 10)
(305)GAAPQPGPAHLSLTITLLMTARLWGGTLLWT(335)

306-335 cleaved to form GPI anchor, 305 (G underlined) is the AA binding the GPI anchor itself (ω)

Functional variants of the disclosed GPI anchor sequences (e.g. the exemplified CD59 GPI anchor sequence) can be used, as will be apparent to the skilled person. The functional variant must retain the function of being successfully cleaved from the larger protein in a host cell during addition of the GPI. This can be readily assessed by a skilled person, for example using the assays described in the Examples to confirm the presence of the GPI-anchored proteins in extracellular vesicles. A comparison may be made, for example, to assess whether varying one or more residues in the anchor sequence varies the result of one of more of the assays in the Examples compared to the native sequence.

A functional GPI anchor sequence may be indirectly determined by observing the shift of proteins from the pellet to the supernatant after treatment of whole cells with PI-PLC. Triton X-114 (a nonionic detergent) phase separation can similarly be used, whereby GPI-anchored proteins partition into the detergent-rich phase before, but not after, PI-PLC treatment.

Typically, a variant of the CD59 GPI anchor sequence has at least 70% sequence identity, at least 80% sequence identity or at least 90% sequence identity to the CD59 GPI anchor sequence used in the Examples and described above.

In some embodiments, the variant of the CD59 GPI sequence contains 1, 2, 3, 4 or 5 substitutions, insertions or deletions relative to the exemplified native sequence.

In some embodiments, the variant of the CD59 GPI sequence contains 1, 2 or 3 substitutions relative to the exemplified native sequence. Typically these are conservative substitutions.

In some embodiments, 1-10 amino acid residues are truncated from the N-terminus of a disclosed GPI sequence, for example the truncation of 1, 2, 3, 4 or 5 amino acids. In some embodiments, 1-10 amino acid residues are truncated from the C-terminus of a disclosed GPI sequence, for example the truncation of 1, 2, 3, 4 or 5 amino acids. In some embodiments, one or more amino acids are truncated from each terminus of the GPI sequence.

A variant GPI anchor sequence (e.g. a variant GP59 anchor sequence) should also fulfil the following three requirements:
(1) three relatively small amino acids (Ala, Asn, Asp, Cys, Gly, or Ser) located at w, ω+1, and ω+2, where w is the amino acid attached to the GPI anchor and where ω+1 and ω+2 are the first two residues of the cleaved peptide;
(2) a relatively polar domain of typically five to 10 residues; and
(3) a hydrophobic domain of typically 15-20 hydrophobic amino acids.

The requirement for entry into the ER for GPI attachment means that the protein to be tethered to GPI will also typically comprise an amino-terminal signal peptide that directs translocation to the ER. This is present, for example, at the N-terminus (residues 1 to 18) of full-length BDNF. If a protein of interest does not comprise an ER-translocation peptide at the N-terminus, then this can be added to the recombinant fusion protein of the invention. N-terminal ER-translocation peptides are very well-known in the art as being around 15-30 amino acids long, typically around 18, 19, 20, 21 or 22 amino acids long. The N-terminal ER-translocation peptides typically comprise primarily hydrophobic residues.

### Glycosylphosphatidylinositol

The GPI moiety that is attached to the protein of the invention comprises the conserved core glycan, phosphatidylinositol (PI), and glycan side chains. The exact structures of mature GPI anchors varies depending on both the protein to which they are attached and the organism in which they are synthesized.

The structure of the core glycan is ethanolamine-PO4-6Manα1-2Manα1-6Manα1-4GlcNα1-6myo-inositol-1-PO4-lipid.

Mammalian GPI is synthesized in the ER. The complete GPI precursor competent for attachment to proteins is synthesized from PI by stepwise reactions before the preassembled GPI is then en bloc transferred to the protein.

The GPI is linked to the C-terminus of the target protein via an amide bond generated between the C-terminal carboxyl group and an amino group of the terminal EtNP.

The entire GPI-anchored protein is then anchored to the outer leaflet of a lipid bilayer by insertion of hydrocarbon chains of PI.

### Linker Sequence

The proteins of the invention can advantageously include a linker region between the N-terminal protein of interest and the C-terminal GPI anchor sequence.

The linker peptide is typically 2 to 20 amino acid residues in length, for example 3 to 15 amino acid residues or 4 to 10 amino acid residues. In some embodiments, the linker peptide is 4, 5, 6 or 7 amino acids in length. In some embodiments, the linker peptide is 6 amino acids in length.

The examples demonstrate that the peptide sequence GAPLE is an effective linker. This may be varied to create a functional variant. A functional variant of this peptide retains the ability to provide an active protein when anchored by GPI in a biological membrane. A functional variant typically includes 1 or 2 substitutions, typically conservative substitutions. Conservative amino acid substitutions are summarised in the table below.

| | | 1-letter |
|---|---|---|
| Class | Amino acids | code |
| Aliphatic | Glycine, Alanine, Valine, | G, A, V, |
| | Leucine, Isoleucine | L, I |
| Hydroxyl or sulfur/ | Serine, Cysteine, Selenocysteine, | S, C, U, |
| selenium-containing | Threonine, Methionine | T, M |
| Cyclic | Proline | P |
| Aromatic | Phenylalanine, Tyrosine, | F, Y, W |
| | Tryptophan | |
| Basic | Histidine, Lysine, Arginine | H, K, R |
| Acidic and their | Aspartate, Glutamate, Asparagine, | D, E, N, |
| amides | Glutamine | |

In some embodiments, the linker can comprise the residues GAPLE but be reordered (e.g. AGELP (SEQ ID NO: 11), AGLEP (SEQ ID NO: 12) or EGLAP (SEQ ID NO: 13)) or reversed (ELPAG (SEQ ID NO: 14)).

In other embodiments, the linker may be a polyglycine linker, optionally comprising or consisting or between 3 and 10 glycine residues. In some embodiments, the linker may comprise or consist of five amino acid residues wherein the third residue is proline. In some embodiments, the linker comprises 5 residues and the fifth (i.e. N-terminal) residue is acidic for example glutamate.

In some embodiments the linker comprises or consists of Xaa Xaa Pro Xaa Glu. Each Xaa is any amino acid residue. Optionally, each Xaa may optionally be an aliphatic residue (G, A, V, L or I; SEQ ID NOs: 15 to 19, respectively). In certain embodiments, each Xaa may optionally be G, A or L.

In some embodiments, the linker comprises or consists of 2 to 20 amino acids selected according to their flexibility where this flexibility is described according to Huang and Nau, 2003 (Angew. Chem. Int. Ed. 2003, 42, 2269-2272, DOI: 10.1002/anie.200250684, incorporated herein by reference in its entirety). For example, the linker may comprise a polypeptide consisting of amino acids with a quenching score of 5 or greater as described in that publication, namely Gly, Ser, Asp, Asn, Ala, Thr, Leu, Phe, Glu, Gln. In some embodiments, the linker comprises or consists of 4 to 12 residues, for example 5, 6, 7 or 8 residues wherein each residue is Gly, Ser, Asp, Asn, Ala, Thr, Leu, Phe, Glu or Gln.

In a further embodiment, the linker domain can be designed to serve as an oligomerisation domain that promotes dimerization or the formation of higher order complexes such as trimers, tetramers or other multimers. Sequences that naturally and preferentially associate are known in the art. For example, multimerisation domains are described by Gupta et al (Nature Biomedical Engineering 1084 VOL 5, September 2021 pages 1084-1098).

In one embodiment, the linker comprises a GCN4-based isoleucine zipper (IZ) domain or a T4 bacteriophage fibritin foldon (Fd) trimerization domain, as described by Sliepen et al (J Biol Chem 2015 Mar. 20; 290 (12): 7436-42). IZ consists of α-helices in a coiled-coil heptad repeat, in which the first (a) and fourth (d) amino acid residues in each heptad repeat determine the oligomerization state of the protein. Isoleucines at the a and d positions facilitate trimerization while alternating isoleucine/leucine or leucine/isoleucine can confer dimerization or tetramerization, respectively, which can be exploited for engineering dimeric, trimeric, or tetrameric proteins. In contrast, Fd consists of three β-hairpins, which assemble into a β-propeller-like structure. The Fd trimer is stabilized by hydrogen-bonding, hydrophobic interactions, and salt-bridges between each.

In another embodiment, the linker comprises a dimerization domain (GCN4 L.Z), derived from yeast as described by Ellenberger et al (Cell. 1992 Dec. 24; 71 (7): 1223-37). The bZIP dimer is a pair of continuous alpha helices that form a parallel coiled coil over their carboxy-terminal 30 residues and gradually diverge toward their amino termini to pass through the major groove of the DNA-binding site. The coiled-coil dimerization interface is oriented almost perpendicular to the DNA axis, giving the complex the appearance of the letter T.

In another embodiment, the linker comprises a tetramerization domain derived from phosphoprotein P (Uniprot: Q9IK91) of the human metapneumovirus as described by Jensen et al (Biophysical Journal 118, 2470-2488 May 19, 2020). Each monomer in this tetramerization domain is composed of two short helices (helices α1 and α2) and a long C-terminal helix (helix α3: aa 508-570 of Uniprot sequence Q9IK91). The long helix of each monomer is composed of eight complete heptad repeats and a stammer at position 540-542, resulting in a kink near residue Pro544. Within one tetramer, the four long helices fold into a parallel coiled coil forming a central shaft, whereas the short helices form a crown around the N-terminal end of this shaft. The structure of this tetramerization domain is provided in PDB accession number 4GJW. In some embodiments, the linker of the invention comprises a multimerisation domain comprising all or a functional portion of residues 476 to 571 of the protein with Uniprot accession no. Q9IK91.

It is also known in the art that protein sequences can be engineered to associate preferentially and form homodimers or heterodimers, such as the knobs-into-holes design of the Fc regions of bispecific antibodies. This design can be applied to the linkers of the present invention to drive preferential dimerization or multimerisation.

### Purification Peptide Sequence

The fusion protein of the first or second aspect may comprise a peptide that simplifies purification, also referred to as a purification tag.

An exemplary purification tag comprises or consists of the sequence YPYDVPDYA or a functional variant thereof. A functional variant may in some embodiments contain 1, 2, 3 or 4 conservative substitutions.

Another suitable purification tag is a polyhistidine tag (e.g. a 6×His tag), as is known in the art to be useful for purifying a protein on a divalent metal ion, most typically nickel, column.

### Extracellular Vesicles

One aspect of the invention provides an extracellular vesicle loaded with a fusion protein of the invention. The protein is anchored into the extracellular vesicle membrane via the GPI.

An extracellular vesicle is a membrane bound vesicle 30 to 1000 nm diameter that is released from a cell. It is limited by a lipid bilayer that encloses biological molecules. The term extracellular vesicle is known in the art and encompasses a number of different species of vesicle, including a membrane particle, membrane vesicle, microvesicle, exosome-like vesicle, exosome, ectosome-like vesicle, ectosome or exovesicle. The different types of microparticle are distinguished based on diameter, subcellular origin, their density in sucrose, shape, sedimentation rate, lipid composition, protein markers and mode of secretion (i.e. following a signal (inducible) or spontaneously (constitutive)). Four of the common microparticles and their distinguishing features are described in Table 1, below.

TABLE 1
Various Microparticles

| Microparticle | Size | Shape | Markers | Lipids | Origin |
|---|---|---|---|---|---|
| Microvesicles | 100-1000 nm | Irregular | Integrins, | Phosphatidylserine | Plasma |
| | | | selectins, | | membrane |
| | | | CD40 ligand | | |
| Exosome-like | 20-50 nm | Irregular | TNFRI | No lipid rafts | MVB from |
| vesicles | | | | | other organelles |
| Exosomes | 30-100 nm; | Cup | Tetraspanins | Cholesterol, | Multivesicular |
| | (<200 nm) | shaped | (e.g. CD63, | sphingomyelin, | endosomes |
| | | | CD9), Alix, | ceramide, lipid rafts, | |
| | | | TSG101, | phosphatidylserine | |
| | | | ESCRT | | |
| Membrane | 50-80 nm | Round | CD133, | Unknown | Plasma |
| particles | | | no CD63 | | membrane |

A typical extracellular vesicle according to the invention is an exosome. Exosomes originate in the endosomal compartment and are secreted when the Multivesicular Bodies (MVBs) fuse with the plasma membrane.

Exosomes are typically CD63+, more typically CD63+ Alix+.

Exosomes were previously typically defined as having a diameter of 30-100 nm, but more recent studies confirm that exosomes can also have a diameter between 100 nm and 200 nm, (e.g. Katsuda, et al. Proteomics 2013, and Katsuda, et al. Scientific Reports 2013). Accordingly, exosomes typically have a diameter between 30 nm and 150 nm. The diameter can be determined by any suitable technique, for example electron microscopy or dynamic light scattering.

Exosomes are thought to play a role in intercellular communication by acting as vehicles between a donor and recipient cell through direct and indirect mechanisms. Direct mechanisms include the uptake of the exosome and its donor cell-derived components (such as proteins, lipids or nucleic acids) by the recipient cell, the components having a biological activity in the recipient cell. Indirect mechanisms include exosome-recipient cell surface interaction and causing modulation of intracellular signalling of the recipient cell. Hence, exosomes may mediate the acquisition of one or more donor cell-derived properties by the recipient cell.

In some embodiments of the invention, the exosomes loaded with a GPI-tagged protein are isolated. The term “isolated” indicates that the exosome or exosome population to which it refers is not within its natural environment. The exosome or exosome population has been substantially separated from surrounding tissue. In some embodiments, the exosome or exosome population is substantially separated from surrounding tissue if the sample contains at least about 75%, in some embodiments at least about 85%, in some embodiments at least about 90%, and in some embodiments at least about 95% exosomes. In other words, the sample is substantially separated from the surrounding tissue if the sample contains less than about 25%, in some embodiments less than about 15%, and in some embodiments less than about 5% of materials other than the exosomes. Such percentage values refer to percentage by weight. The term encompasses exosomes that have been removed from the organism from which they originated and exist in culture. The term also encompasses exosomes that have been removed from the organism from which they originated, and subsequently re-inserted into an organism.

The extracellular vesicles loaded with the GPI-tethered protein of the invention can be produced by expressing the fusion protein of the invention containing the GPI anchor sequence in a host cell, for example a neural stem cell such as CTX0E03. The expression will typically comprise introducing into the host cell a nucleic acid expression vector encoding the protein of the invention. The vector will typically comprise an inducible or constitutive promoter.

A method of the invention discloses loading of exosomes with exogenous proteins anchored in their membranes by introducing into the host cell an exogenous recombinant nucleic acid encoding the fusion protein of the invention containing the GPI anchor sequence.

Extracellular vesicles such as exosomes can be secreted from virtually any cell type. In some embodiments extracellular vesicles such as exosomes are secreted from stem cells, hIPSCs, tissue stem cells, differentiated cells from any of those or dendritic cells. In a certain embodiment, the Extracellular vesicles such as exosomes are secreted from stem cells. Stem cells naturally produce exosomes by the fusion of intracellular multivesicular bodies (which contain microparticles) with the cell membrane and the release of the exosomes into the extracellular compartment.

In another embodiment, the stem cell is a neural stem cell. Neural stem cells (NSCs) are self-renewing, multipotent stem cells that generate neurons, astrocytes and oligodendrocytes (Kornblum, 2007). In some embodiments, the neural stem cell line may be the “CTX0E03” cell line, the “STR0C05” cell line, the “HPC0A07” cell line or the neural stem cell line disclosed in Miljan et al. 2009. The neural stem cell may be any of the neural stem cells described herein, for example the CTX0E03 conditionally-immortalised cell line, which is clonal, standardised, shows clear safety in vitro and in vivo and can be manufactured to scale thereby providing a unique resource for stable exosome production. Alternatively, the neural stem cells may be neural retinal stem cell lines, optionally as described in U.S. Pat. No. 7,514,259 (which is incorporated by reference).

A neural stem cell exosome is an exosome that is produced by a neural stem cell. Typically, the exosome is secreted by the neural stem cell. Exosomes from other cells, such as mesenchymal stem cells, are known in the art.

A neural stem cell that produces the exogenous nucleotide sequence loaded exosomes of the invention can be a fetal, an embryonic, or an adult neural stem cell, such as has been described in U.S. Pat. Nos. 5,851,832, 6,777,233, 6,468,794, 5,753,506 and WO-A-2005121318 (incorporated by reference). The fetal tissue may be human fetal cortex tissue. The cells can be selected as neural stem cells from the differentiation of induced pluripotent stem (iPS) cells, as has been described by Yuan, et al. (2011) or a directly induced neural stem cell produced from somatic cells such as fibroblasts (for example by constitutively inducing Sox2, Klf4, and c-Myc while strictly limiting Oct4 activity to the initial phase of reprogramming as recently by Their, et al. 2012). Human embryonic stem cells may be obtained by methods that preserve the viability of the donor embryo, as is known in the art (e.g. Klimanskaya et al. 2006, and Chung, et al. 2008). Such non-destructive methods of obtaining human embryonic stem cell may be used to provide embryonic stem cells from which microparticles of the invention can be obtained. Alternatively, the exogenous nucleotide sequence loaded exosomes of the invention can be obtained from adult stem cells, iPS cells or directly-induced neural stem cells. Accordingly, the exogenous nucleotide sequence loaded exosomes of the invention can be produced by multiple methods that do not require the destruction of a human embryo or the use of a human embryo as a base material.

In further embodiments, the cell can be a pluripotent stem cell, for example an induced pluripotent stem cell (iPSC). The cell can also be a cell that is differentiated from an iPSC, which may be a stem cell derived from an iPSC or a terminally-differentiated cell derived from an iPSC. Cells derived from an iPSC may be of the endoderm, mesoderm or ectoderm lineage.

In some embodiments, iPSCs and their progeny can be derived from conditionally immortalised stem cells, typically conditionally-immortalised neural stem cells. Accordingly, in some embodiments, the host cell is an iPSC comprising the c-mycER transgene, optionally in its genome. The iPSCs may be reprogrammed CTX0E03 cells, as described in WO-A-2020074925 and PCT/GB2021/050905 (both incorporated herein by reference in its entirety). In one embodiment, therefore, the host cell used according to the invention is a CTX0E03 cell that that has been reprogrammed to pluripotency and is therefore an iPSC. Reprogramming typically involves introducing one or more transcription factors into the cell to be reprogrammed. In some embodiments, the transcription factors comprise or consist of: OCT4 and SOX2; OCT, KLF4 and SOX2; or OCT4, KLF4, SOX2 and MYC. Cells derived from those CTX0E03-iPSCs that can be used according to the invention include cells of the haematopoietic lineage.

Typically, the cell population from which the exosomes are produced is substantially pure. The term “substantially pure” as used herein, refers to a population of cells that is at least about 75%, in some embodiments at least about 85%, in some embodiments at least about 90%, and in some embodiments at least about 95% pure, with respect to other cells that make up a total cell population. For example, with respect to neural stem cell populations, this term means that there are at least about 75%, in some embodiments at least about 85%, in some embodiments at least about 90%, and in some embodiments at least about 95% pure, neural stem cells compared to other cells that make up a total cell population. In other words, the term “substantially pure” refers to a population of stem cells of the present invention that contain fewer than about 25%, in some embodiments fewer than about 15%, and in some embodiments fewer than about 5%, of lineage committed cells in the original unamplified and isolated population prior to subsequent culturing and amplification.

An exosome comprises at least one lipid bilayer which typically encloses a milieu comprising lipids, proteins and nucleic acids. The nucleic acids may be deoxyribonucleic acid (DNA) and/or ribonucleic acid (RNA). RNA may be messenger RNA (mRNA), micro RNA (miRNA) or any miRNA precursors, such as pri-miRNA, pre-miRNA, and/or small nuclear RNA (snRNA).

A stem cell-derived exosome typically retains at least one biological function of the stem cell from which it is derived. Biological functions that may be retained include the ability to promote angiogenesis and/or neurogenesis, the ability to effect cognitive improvement in the brain of a patient that has suffered a stroke, or the ability to accelerate blood flow recovery in peripheral arterial disease. For example, CTX0E03 cells are known to inhibit T cell activation in a PBMC assay and, in one embodiment, the microparticles of the invention retain this ability to inhibit T cell activation in a PBMC assay. PBMC assays are well-known to the skilled person and kits for performing the assay are commercially available.

Some exosomes of the invention express the CD133 surface marker. Other exosomes of the invention do not express the CD133 surface marker. “Marker” refers to a biological molecule whose presence, concentration, activity, or phosphorylation state may be detected and used to identify the phenotype of a cell.

Exosomes are endosome-derived lipid microparticles of typically 30-150 nm diameter and sometimes between 100 nm and 200 nm diameter that are released from the cell by exocytosis. Exosome release occurs constitutively or upon induction, in a regulated and functionally relevant manner. During their biogenesis, exosomes incorporate a wide range of cytosolic proteins (including chaperone proteins, integrins, cytoskeletal proteins and the tetraspanins) and genetic material. Consequently, exosomes are considered to be inter-cellular communication devices for the transfer of proteins, lipids and genetic material between cells, in the parent cell microenvironment and over considerable distance. Although the invention is not bound by this theory, it is possible that the exosomes are responsible for the efficacy of the neural stem cells. Therefore, exosomes from neural stem cells are themselves expected to be therapeutically efficacious.

In one embodiment, isolated or purified exogenous nucleotide sequence loaded exosomes are also loaded with one or more exogenous nucleic acids, lipids, proteins, drugs or prodrugs which are intended to perform a desired function in a target cell. This does not require manipulation of the stem cell and the exogenous material can optionally be directly added to the exosomes. For example, exogenous peptides or proteins can be introduced into the exosomes by electroporation. The microparticles can then be used as vehicles or carriers for the exogenous material. In this way, microparticles can be used as vehicles to deliver one or more agents, typically therapeutic or diagnostic agents, to a target cell.

### Neural Stem Cells

In some embodiments, the host cell that expresses the protein of the invention and produced loaded extracellular vesicles according to the invention is a neural stem cell.

The neural stem cell may be a stem cell line, i.e. a culture of stably dividing stem cells. A stem cell line can to be grown in large quantities using a single, defined source. Immortalisation may arise from a spontaneous event or may be achieved by introducing exogenous genetic information into the stem cell which encodes immortalisation factors, resulting in unlimited cell growth of the stem cell under suitable culture conditions. Such exogenous genetic factors may include the gene “myc”, which encodes the transcription factor Myc. The exogenous genetic information may be introduced into the stem cell through a variety of suitable means, such as transfection or transduction. For transduction, a genetically engineered viral vehicle may be used, such as one derived from retroviruses, for example lentivirus.

Additional advantages can be gained by using a conditionally immortalised stem cell line, in which the expression of the immortalisation factor can be regulated without adversely affecting the production of therapeutically effective microparticles. This may be achieved by introducing an immortalisation factor which is inactive unless the cell is supplied with an activating agent. Such an immortalisation factor may be a gene such as c-mycER. The c-MycER gene product is a fusion protein comprising a c-Myc variant fused to the ligand-binding domain of a mutant estrogen receptor. c-MycER only drives cell proliferation in the presence of the synthetic steroid 4-hydroxytamoxifen (4-OHT) (Littlewood, et al. 1995). This approach allows for controlled expansion of neural stem cells in vitro, while avoiding undesired in vivo effects on host cell proliferation (e.g. tumour formation) due to the presence of c-Myc or the gene encoding it in microparticles derived from the neural stem cell line. A suitable c-mycER conditionally immortalized neural stem cell is described in U.S. Pat. No. 7,416,888. The use of a conditionally immortalised neural stem cell line therefore provides an improvement over existing stem cell microparticle isolation and production.

Preferred conditionally-immortalised cell lines include the CTX0E03, STR0C05 and HPC0A07 neural stem cell lines, which have been deposited by the present applicant at the European Collection of Animal Cultures (ECACC), Vaccine Research and Production laboratories, Public Health Laboratory Services, Porton Down, Salisbury, Wiltshire, SP4 0JG, with Accession No. 04091601 (CTX0E03); Accession No. 04110301 (STROC05); and Accession No. 04092302 (HPC0A07). The derivation and provenance of these cells is described in EP1645626 B1. The advantages of these cells are retained by exosomes produced by these cells.

The cells of the CTX0E03 cell line may be cultured in the following culture conditions:
Human Serum Albumin 0.03%
Human Transferrin 5 μg/ml
Putrescine Dihydrochloride 16.2 μg/ml
Human recombinant Insulin 5 μ/ml
Progesterone 60 ng/ml
L-Glutamine 2 mM
Sodium Selenite (selenium) 40 ng/ml

Plus basic Fibroblast Growth Factor (10 ng/ml), epidermal growth factor (20 ng/ml) and 4-hydroxytamoxifen 100 nM for cell expansion. The cells can be differentiated by removal of the 4-hydroxytamoxifen. Typically, the cells can be cultured at 5% CO₂/37° C. These cell lines do not require serum to be cultured successfully. Serum is required for the successful culture of many cell lines, but contains many contaminants including its own exosomes. A further advantage of the CTX0E03, STR0C05 or HPC0A07 neural stem cell lines, or any other cell line that does not require serum, is that the contamination by serum is avoided.

The cells of the CTX0E03 cell line (and microparticles derived from these cells) are multipotent cells originally derived from 12 week human fetal cortex. The isolation, manufacture and protocols for the CTX0E03 cell line is described in detail by Sinden, et al. (U.S. Pat. No. 7,416,888 and EP1645626 B1). The CTX0E03 cells are not “embryonic stem cells”, i.e. they are not pluripotent cells derived from the inner cell mass of a blastocyst; isolation of the original cells did not result in the destruction of an embryo.

CTX0E03 is a clonal cell line that contains a single copy of the c-mycER transgene that was delivered by retroviral infection and is conditionally regulated by 4-OHT (4-hydroxytamoxifen). The c-mycER transgene expresses a fusion protein that stimulates cell proliferation in the presence of 4-OHT and therefore allows controlled expansion when cultured in the presence of 4-OHT. This cell line is clonal, expands rapidly in culture (doubling time 50-60 hours) and has a normal human karyotype (46 XY). It is genetically stable and can be grown in large numbers. The cells are safe and non-tumorigenic. In the absence of growth factors and 4-OHT, the cells undergo growth arrest and differentiate into neurons and astrocytes.

The development of the CTX0E03 cell line has allowed the scale-up of a consistent product for clinical use. Production of cells from banked materials allows for the generation of cells in quantities for commercial application (Hodges, et al. 2007).

The term “culture medium” or “medium” is recognized in the art, and refers generally to any substance or preparation used for the cultivation of living cells. The term “medium”, as used in reference to a cell culture, includes the components of the environment surrounding the cells. Media may be solid, liquid, gaseous or a mixture of phases and materials. Media include liquid growth media as well as liquid media that do not sustain cell growth. Media also include gelatinous media such as agar, agarose, gelatin, collagen matrices and/or other proteins forming any extracellular matrix. Exemplary gaseous media include the gaseous phase to which cells growing on a petri dish or other solid or semisolid support are exposed. The term “medium” also refers to material that is intended for use in a cell culture, even if it has not yet been contacted with cells. In other words, a nutrient rich liquid prepared for bacterial culture is a medium. Similarly, a powder mixture that when mixed with water or other liquid becomes suitable for cell culture may be termed a “powdered medium”. “Defined medium” refers to media that are made of chemically defined (usually purified) components. “Defined media” do not contain poorly characterized biological extracts such as yeast extract and beef broth. “Rich medium” includes media that are designed to support growth of most or all viable forms of a particular species. Rich media often include complex biological extracts. A “medium suitable for growth of a high density culture” is any medium that allows a cell culture to reach an OD600 of 3 or greater when other conditions (such as temperature and oxygen transfer rate) permit such growth. The term “basal medium” refers to a medium which promotes the growth of many types of microorganisms which do not require any special nutrient supplements. Most basal media generally comprise of four basic chemical groups: amino acids, carbohydrates, inorganic salts, and vitamins. A basal medium generally serves as the basis for a more complex medium, to which supplements such as serum, buffers, growth factors, lipids, and the like are added. In one aspect, the growth medium may be a complex medium with the necessary growth factors to support the growth and expansion of the cells of the invention while maintaining their self-renewal capability. Examples of basal media include, but are not limited to, Eagles Basal Medium, Minimum Essential Medium, Dulbecco's Modified Eagle's Medium, Medium 199, Nutrient Mixtures Ham's F-10 and Ham's F-12, McCoy's 5A, Dulbecco's MEM/F-I 2, RPMI 1640, and Iscove's Modified Dulbecco's Medium (IMDM).

### Transfection of Cells to Produce Extracellular Vesicles

Transfection of the extracellular vesicle (e.g. exosome) producer cells with the nucleic acid construct of the invention can be carried out using various methods known in the art. Such methods include, but are not limited to, cationic lipid transfection, electroporation, viral transfection, and calcium phosphate transfection. The Examples below use a lentiviral expression system.

A nucleic acid construct of the invention comprises sequence encoding a protein of the invention.

In some embodiments, cationic lipid transfection is used to transfect the nucleic acid construct into the exosome producer cell. In a further embodiment, cationic lipid transfection is carried out using reagents such as, DOTMA (N-[1-(2,3,-dioleyloxy) propyl]-N, N, N-trimethylammonium chloride), TransIT®, X-tremeGENE™ transfection reagent Lipofectin®, Lipofectamine®, and Oligofectamine®.

In some embodiments, electroporation is used to transfect the nucleic acid construct into the exosome producer cell. Electroporation involves exposure of cell membranes to high-intensity electric pulses which cause temporary destabilisation making the cell highly permeable, allowing the entry of exogenous molecules. Electroporation is an easy, non-chemical technique that can yield high transformation efficiencies in various cell types.

In some embodiments, viral transfection is used to transfect the nucleic acid construct into the exosome producer cell. This method involves the use of viral vectors to deliver nucleic acids into cells. Viral delivery systems such as lentiviral, adenoviral, adeno-associated viral systems and oncoretroviral vectors can be used for transferring nucleotide sequences, even in hard-to-transfect cells.

In some embodiments, calcium phosphate is used to transfect the nucleic acid construct into the exosome producer cell. The calcium phosphate transfection technique involves the precipitation of DNA and calcium phosphate. The precipitation is facilitated by mixing a HEPES-buffered saline solution, having sodium phosphate, with calcium chloride solution and DNA2.

The amount of transfected nucleic acid construct present in a purified exosomes can be quantified using qPCR (Example 1) or other techniques that allow RNA quantification such as FISH/Scope, droplet-digital PCR or RNAseq.

### Exosome Purification

Exosomes of the invention may be purified using known exosome purification techniques. For example, exosomes can be purified by Tangential Flow Filtration (TFF) or ultracentrifugation, e.g. 100000×g for 1-2 hours. Alternative or additional methods for purification of may be used, such as antibody-based methods, e.g. immunoprecipitation, magnetic bead purification, resin-based purification, using specific antibodies.

The exosomes can be subsequently quantified and characterised as described in WO-A-2013/150303 and WO-A-2014/013258 (incorporated by reference).

Packaging of Exogenous Nucleotide Sequences within EVs for Delivery to Target Cells

Extracellular vesicles such as exosomes represent a particularly interesting delivery option for the delivery of exogenous protein sequences to target cells. The present invention capitalises on an endogenous system for intercellular communication thereby providing a system for the delivery of exogenous proteins into target cells. Exosomes can be modified to target a variety of specific cell types and tissues. Exosomes secreted from different cell types express different proteins on their surface that may target them to different target cells.

The invention therefore provides an exosome loaded an exogenous protein cargo, for example an exosome loaded with a therapeutic protein, by anchoring the exogenous protein cargo into the exosome membrane. The invention also provides a method of delivering a protein to a target cell, using an exosome loaded with the GPI-anchored protein, wherein the method comprises contacting the target cell with the loaded exosomes. In some embodiments, a targeting moiety is expressed or conjugated to the surface of the exosomes to target the exosome comprising an exogenous nucleotide sequence to a particular cell type.

A target cell is a cell in which the exogenous nucleotide sequence is intended to modulate a biological activity e.g. gene activity or receptor signalling activity. A target cell may be a cell in vitro or in vivo. In some embodiments the target cell is a cancer cell, a stem cell, an immune cell. In other embodiments, the target cell is a neuronal cell, a stromal cell, or a muscle cell. In some embodiments, the target cell is a cell of the CNS.

### Assessing Gene Modulation in the Target Cell

There are many methods that can be used to assess modulation of gene activity in a target cell. The following methods are to serve as examples of methods and are not limiting. In some embodiments, the pre-miRNA of the invention results in a reduction of expression a target gene in a target cell.

Western blot is a widely used analytical technique used in molecular biology to detect specific protein molecules from a mixture of proteins. Western blotting can be used to measure the amount of protein expression. The method includes, preparing the protein sample by mixing it with a detergent, such as SDS, separation of the proteins using gel electrophoresis, transferring the proteins from the gel to a blotting membrane, blocking the membrane, incubating with a primary antibody, incubating with secondary antibody linked to a reporter enzyme that produces colour or light, and detecting this colour or light. In some embodiments quantitative western blotting can be carried out to assess modulation of gene activity. In some embodiments, a reduction in protein quantity is indicative of down-regulation of a gene. In another embodiment, an increase in protein quantity is indicative of up-regulation of a gene.

Changes in gene expression in cells can be measured using quantitative polymerase chain reaction (PCR). In some embodiments, quantitative PCR (qPCR) can be carried out to assess modulation of gene activity. Such a method may include isolating total RNA from the target cell, performing cDNA synthesis, running qPCR reactions, and analysing the results from qPCR using relative quantification (Fleige and Pfaffl. 2006). In some embodiments, the qPCR is carried out using SYBR-green or TaqMan/TaqPath probes. In some embodiments, a reduction in RNA is indicative of down-regulation of a gene. In another embodiment, an increase in RNA is indicative of up-regulation of a gene.

In some embodiments, reporter assays are used to assess gene modulation. In a further embodiment, the reporter assay is a fluorescence reporter assay using a fluorescent protein such as tagBFP, GFP, eGFP, YFP, mcherry, Ruby2, mOrange, Citrine, Clover, and mTurquoise. A fluorescent protein reporter system is described in Example 1, and FIGS. 5 and 6. Changes in expression of the fluorescence protein or fusion protein can be measured using flow cytometry, microscopy, or high throughput quantitative microscopy. In some embodiments, a reduction in fluorescent signal is indicative of down-regulation of a gene. In another embodiment, an increase in fluorescent signal is indicative of up-regulation of a gene.

In another embodiment, the reporter assay uses a luciferase-based system. In some embodiments, the luciferase reporter that can be used is Cypridina Luciferase, Gaussia Luciferase, Gaussia-Dura Luciferase, Green Renilla Luciferase, Red Firefly Luciferase, Renilla Luciferase, Nano-Luc Luciferase or TurboLuc Luciferase. In the luciferase-based system a change in gene expression is measured using a luminometer or modified optical microscopes (McClure, et al. 2011), but can also been measured by other means such as qPCR, Western Blot, immunochemistry or flow cytometry. In some embodiments, a reduction in signal is indicative of down-regulation of a gene. In another embodiment, an increase in signal is indicative of up-regulation of a gene.

In some embodiments of the invention, the nucleic acid construct of the invention results in a reduction of gene expression by at least 10%, 20%, 30%, 40%, 50%, 60%, 70%, 80%, 90% or greater. Typically, gene expression is reduced by 60% to 100%. In some embodiments, of the invention, the nucleic acid construct of the invention results in an increase in gene expression by at least 10%, 20%, 30%, 40%, 50%, 60%, 70%, 80%, 90% or greater. Typically, gene expression is increased by 60% to 100%.

### Therapeutic Uses

Extracellular vesicles loaded with protein may be useful in the treatment or prophylaxis of disease. Accordingly, the invention includes a method of treating or preventing a disease or disorder in a patient using an EV such as an exosome loaded with protein anchored in the exosome membrane via CPI. The term “patient” includes human and other mammalian subjects that receive either prophylactic or therapeutic treatment.

The invention also provides a method for treating or preventing a disease or condition comprising administering an effective amount of the exosome of the invention, thereby treating or preventing the disease. The exosomes of the invention can be used to treat the same diseases as the stem cells from which they are obtained.

In prophylactic applications, pharmaceutical compositions or medicaments are administered to a patient susceptible to, or otherwise at risk of, a particular disease in an amount sufficient to eliminate or reduce the risk or delay the outset of the disease. In therapeutic applications, compositions or medicaments are administered to a patient suspected of, or already suffering from such a disease in an amount sufficient to cure, or at least partially arrest, the symptoms of the disease and its complications. An amount adequate to accomplish this is defined as a therapeutically- or pharmaceutically-effective dose. In both prophylactic and therapeutic regimes, agents are typically administered in several dosages until a sufficient response has been achieved. Typically, the response is monitored and repeated dosages are given if the response starts to fade.

Effective doses of the compositions of the present invention, for the treatment of the above described conditions vary depending upon many different factors, including means of administration, target site, physiological state of the patient, whether the patient is human or an animal, other medications administered, and whether treatment is prophylactic or therapeutic.

As used herein, the terms “treat”, “treatment”, “treating” and “therapy” when used directly in reference to a patient or subject shall be taken to mean the amelioration of one or more symptoms associated with a disorder, or the prevention or prophylaxis of a disorder or one or more symptoms associated with a disorder. The disorders to be treated include, but are not limited to, a degenerative disorder, a disorder involving tissue destruction, a neoplastic disorder, an inflammatory disorder, an autoimmune disease or an immunologically mediated disease including rejection of transplanted organs and tissues. Amelioration or prevention of symptoms results from the administration of the microparticles of the invention, or of a pharmaceutical composition comprising these microparticles, to a subject in need of said treatment.

The exosomes loaded with protein and methods of the invention may be used in the treatment of a proliferative disease. The term ‘proliferative disease’ as used herein refers to both cancer and non-cancer disease. As such, the methods may ultimately result in the killing of cells which proliferate abnormally, such as cancerous cells, including tumour cells, and other (non-malignant) tumour cells. The invention promotes the packaging of therapeutic protein onto exosomes, which are then delivered to the cell of a patient.

In certain embodiments, the treatment is the treatment or prevention of a central nervous system disease or disorder, optionally Huntington's Disease, Alzheimer's Disease, Multiple Sclerosis, Parkinson's Disease, Rett Syndrome or Amyotrophic Lateral Sclerosis.

In other embodiments the treatment is of an injury, disorder or disease of the eye. An ophthalmic condition may involve the loss of functional cells such as retinal cells. In some embodiments, the ophthalmic indication is retinitis pigmentosa or glaucoma. Other diseases of the eye that can be treated according to the present invention include wet macular degeneration, dry macular degeneration, dry eye disease, diseases or disorders of the retina, or diseases or disorders of the cornea. In some embodiments a disorder of the eye may be treated by administering loaded exosomes of the invention directly to the eye. This may in some embodiments be topical administration to the cornea. Such topical administration can conveniently be provided by an eye-drop formulation. In other embodiments, the eye may be treated by injection into the eye ball, typically intravitreal injection.

The term “patient” includes human and other mammalian subjects that receive either prophylactic or therapeutic treatment.

The exosomes loaded with protein of the invention may optionally be combined with another therapeutic agent to provide a combination therapy.

### Pharmaceutical Compositions

The EV's e.g. exosomes loaded with protein are useful in therapy and can therefore be formulated as a pharmaceutical composition. A pharmaceutically acceptable composition typically includes at least one pharmaceutically acceptable carrier, diluent, vehicle and/or excipient in addition to the exosomes of the invention. An example of a suitable carrier is Ringer's Lactate solution. A thorough discussion of such components is provided in Gennaro (2000) Remington: The Science and Practice of Pharmacy. 20th edition, ISBN: 0683306472.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The composition, if desired, can also contain minor amounts of pH buffering agents. The carrier may comprise storage media such as Hypothermosol®, commercially available from BioLife Solutions Inc., USA. Examples of suitable pharmaceutical carriers are described in “Remington's Pharmaceutical Sciences” by E W Martin. Such compositions will contain a prophylactically or therapeutically effective amount of a prophylactic or therapeutic exosome preferably in purified form, together with a suitable amount of carrier so as to provide the form for proper administration to the subject. The formulation should suit the mode of administration. In a preferred embodiment, the pharmaceutical compositions are sterile and in suitable form for administration to a subject, preferably an animal subject, more preferably a mammalian subject, and most preferably a human subject.

The pharmaceutical composition of the invention may be in a variety of forms. These include, for example, semi-solid, and liquid dosage forms, such as lyophilized preparations, liquid solutions or suspensions, injectable and infusible solutions. The pharmaceutical composition is preferably injectable.

Pharmaceutical compositions will generally be in aqueous form. Compositions may include a preservative and/or an antioxidant.

To control tonicity, the pharmaceutical composition can comprise a physiological salt, such as a sodium salt. Sodium chloride (NaCl) is preferred, which may be present at between 1 and 20 mg/ml. Other salts that may be present include potassium chloride, potassium dihydrogen phosphate, disodium phosphate dehydrate, magnesium chloride and calcium chloride.

Compositions may include one or more buffers. Typical buffers include: a phosphate buffer; a Tris buffer; a borate buffer; a succinate buffer; a histidine buffer; or a citrate buffer. Buffers will typically be included at a concentration in the 5-20 mM range. The pH of a composition will generally be between 5 and 8, and more typically between 6 and 8 e.g. between 6.5 and 7.5, or between 7.0 and 7.8.

The composition is preferably sterile. The composition is preferably gluten free. The composition is preferably non-pyrogenic.

In a typical embodiment, the exogenous nucleotide sequence loaded exosomes of the invention are suspended in a composition comprising 6-hydroxy-2,5,7,8-tetramethylchroman-2-carboxylic acid (Trolox®), Na⁺, K⁺, Ca²⁺, Mg²⁺, Cl⁻, H₂PO₄⁻, HEPES, lactobionate, sucrose, mannitol, glucose, dextron-40, adenosine and glutathione. Typically, the composition will not include a dipolar aprotic solvent, e.g. DMSO. Suitable compositions are available commercially, e.g. HypoThermasol®-FRS. Such compositions are advantageous as they allow the exosomes to be stored at 4° C. to 25° C. for extended periods (hours to days) or preserved at cryothermic temperatures, i.e. temperatures below −20° C. The exosomes may then be administered in this composition after thawing.

The pharmaceutical composition can be administered by any appropriate route, which will be apparent to the skilled person depending on the disease or condition to be treated. Typical routes of administration include intravenous, intra-arterial, intramuscular, subcutaneous, intracranial, intranasal or intraperitoneal.

The exosomes of the invention will be administered at a therapeutically or prophylactically-effective dose, which will be apparent to the skilled person. Due to the low or non-existent immunogenicity of the exosomes, it is possible to administer repeat doses without inducing a deleterious immune response.

The disclosure is illustrated by the following non-limiting Examples. It is understood that the Examples and embodiments described herein are for illustrative purposes only and that various modifications or changes in light thereof will be suggested to persons skilled in the art and are to be included within the spirit and purview of this application and scope of the appended claims. All publications, sequence accession numbers, patents, and patent applications cited herein are hereby incorporated by reference in their entirety for all purposes. The terminology used herein is for the purpose of describing particular embodiments only and is not intended to be limiting of the invention. As used herein, the term “and/or” includes any and all combinations of one or more of the associated listed items. As used herein, the singular forms “a”, “an”, and “the” are intended to include the plural forms as well as the singular forms, unless the context clearly indicates otherwise.

The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefits and each can also be used in conjunction with one or more, or in some cases all, of the other disclosed techniques. Accordingly, for the sake of clarity, this description refrains from repeating every possible combination of the individual steps in an unnecessary fashion. Nevertheless, the specification and claims should be read with the understanding that such combinations are entirely within the scope of the invention and the claims.

### Examples

### Summary of the Examples

The Examples investigate the feasibility of presenting neuroprotective growth factors to neural cells in the context of an exosome. Exosomes are nanoscale lipid vesicles generated by all cells and are believed to play an important role in intercellular communication. Presenting the growth factors in the context of an exosome may confer several advantages over native growth factor alone including multivalent presentation to cell-surface receptors, improved distribution and crossing of natural barriers (e.g the blood brain barrier or the blood-cerebrospinal fluid barrier) and improved stability of the payload.

More specifically, the inventors designed and developed a system to express the fusion protein BDNF-HA on the exosomal surface using the GPI sequence from the exosomal protein CD59. To achieve this, the full CDS for the human BDNF was fused to a HA-tag (to enable detection) followed by a short amino acid linker sequence (to add flexibility and separation) followed by the predicted GPI sequence of the CD59 human protein (to confer loading onto lipid membranes including exosomes), a T2A sequence (to allow self-cleaving of the blasticidin resistance marker protein) and a blasticidin resistance gene. Lentiviral particles generated using that construct were used to transduce CTX cells and transduced cells were clonally selected by treatment with blasticidin. The exosomes produced by those cells (referred to herein as Exo-BDNF) were isolated and purified from conditioned media from transduced cell cultures using size exclusion chromatography and tangential flow filtration.

The activity of BDNF in Exo-BDNF was assessed using a variety of in vitro assays, including confirmation that Exo-BDNF binds to the extracellular part of the high affinity receptor for BDNF, TrkB; that Exo-BDNF activates the MAPK signaling pathway in cells expressing TrkB and that Exo-BDNF activates transcription of the immediate-early BDNF-target gene c-fos in target cells.

The biological activity of Exo-BDNF in vivo is also assessed. Using intra-thecal administration (eg through the cerebrospinal fluid circulation) in murine hosts, we compared the ability of the following preparations to activate the BDNF target gene c-fos: vehicle control, exosomes alone, recombinant BDNF alone (rBDNF) and Exo-BDNF. Brains from treated mice were harvested at 2 hr and 6 hr after administration and c-fos mRNA levels in the cortex, striatum and hippocampus were analysed by relative qRT-PCR. For this analysis, expression of TATA-binding protein (TBP) and 18S were used as reference genes. The concentration of BDNF expressed by Exo-BDNF is also reported using a quantitative western blotting approach developed during this work.

A commercially-available reporter assay confirmed that ExoBDNF activates TrkB signalling suggesting activity which is not present in unmodified exosome, thereby indicating BDNF-specific activity in ExoBDNF preparations.

Finally, a pro-survival effect of ExoBDNF on rat retinal ganglion cells (RGC) is demonstrated, suggesting that ExoBDNF confers an advantage in survival of neurite-bearing RGC after mechanical insult.

### Materials and Methods

### Cells

HEK293T cells (ATCC) were grown in DMEM media supplemented with 10% Bovine serum, pen/strep (100 units/ml) and 1% non-essential amino acids. Cells were kept in an incubator with 5% CO₂, 37° C. and H₂O saturated atmosphere. ReNeuron's cortex-derived neural stem cell progenitors (CTX0E03) were cultured as previously described. Where appropriate, transfected or transduced cells were incubated in culture with 1-10 μg/mL blasticidin to select for cells expressing the ectopic construct.

### Plasmids and Constructs for ExoBDNF Expression

For stable transduction and ectopic expression, the DNA constructs were synthesized and subsequently cloned into a pLVX lentiviral vector plasmid. Lentiviral particles were generated using standard protocols and producer lines as described elsewhere. For transient expression, the BDNF construct mentioned above was cloned into a pCDNA3.1-based expression vector and transfected into target cells. Briefly, HEK293T cells were seeded on 15 cm plates (9×10⁶ cells/plate) and transfected on the next day. DNA (10 μg) was added to 250 μl of OptiMEM and then 35 μl of Polyethylenimine at 1 ug/uL (Polysciences #23966-2) (DNA:PEI ratio 1:3) was added and mixed pipetting up and down for 10 times. The mixture was incubated for 20 minutes at room temperature and then added to the cells for 2 days.

### Exosome Isolation

Conditioned media was obtained from cell cultures either transiently or stably expressing BDNF fusion constructs described above or control constructs. Conditioned media was subjected to initial clearing by low-speed centrifugation, followed by volume reduction and dialfiltration with PBS using a tangential flow filtration process. The filtered product was then purified further by size exclusion chromatography and stored at 4′C prior to use.

### TrkB Binding Assay by Flow Cytometry

The antigen-binding domain of TrkB (BDNF receptor) was conjugated to magnetic Dynabeads, and these beads were subsequently incubated with samples of exosome prepared as above, according to the bead manufacturer's recommendations. Subsequently, beads were counterstained with anti-CD81 antibodies and CD81-positive beads were measured by flow cytometry.

### MAPK Pathway Activation and CREB Reporter Assays

For MAPK pathway activation, SH-SY5Y cells were treated with native exosomes or Exo-BDNF and protein lysates were prepared using standard procedures and lysates were probed for total and phosphorylated ERK protein by Western blot.

HEK293T cells were stably transduced with constructs encoding a functional TrkB receptor and a CREB-responsive luciferase reporter cassette. Subsequently, these transduced cells were treated with native exosomes or Exo-BDNF before analysis. For CREB reporter activation, cells were seeded into culture dishes prior to treatment and following treatment, luciferase luminescence was measured using a luminescence plate reader.

### Measurement of Neurite Outgrowth in Differentiated SH-SY5Y Cells

SH-SY5Y cells were differentiated into BDNF-sensitive dopaminergic neurons using a protocol described elsewhere. Once established, cultures of dopaminergic neurons derived from SH-SY5Y cells were treated with native exosomes or Exo-BDNF and morphological changes including neurite growth was monitored using the Incucyte Zoom system and a neurite outgrowth algorithm.

### Western Blot Analyses

Exo-Pro and Exo-BDNF samples (200 uL) obtained from ReNeuron were added to 60 μl of 5× Laemmli buffer and boiled for 15 minutes with lid open to concentrate proteins in a fume hood. Following this, proteins were resolved in 10% Bis-Tris acrylamide gels. Samples were run in MES buffer at 100 V for 1 hour and then transferred to PVDF membranes. Later on, membranes were blocked with TBST+3% BSA (Sigma #A7096) for at least 30 minutes at room temperature and incubated with antibodies for HA (Roche #11583816001) or BDNF (Icosagen #327-100) overnight at 4° C. Next day, (all steps were performed at room temperature) membranes were washed 3 times for 10 minutes with TBS-T, blocked with TBS-T containing 5% non-fat milk at room temperature for 10 minutes and then added anti-mouse-HRP antibodies (Jackson Laboratories #115-035-003) at 1:7,500 dilution to the blocking solution and incubated for 40 minutes. Finally, the membranes were washed again 3 times for 10 minutes with TBST and developed with Western Bright ECL (Advansta K-12045) diluted 1:2 with H₂O. The signal was detected using a McroChemi chemiluminescence apparatus (Bio-Imaging Systems).

### In Vivo Administration of Exo-BDNF in Murine Recipients and Harvest of Brain Tissue

(Use CRO report for more detail here). Briefly, male BALB/c mice at 5-7 weeks old were treated with a single intrathecal injection of either native exosomes, ExoBDNF, recombinant BDNF or vehicle control, and were euthanised 2 h or 6 h post-treatment. Whole brains were extracted immediately and snap-frozen in LN2 and then stored at −80° C. until processed. On the day of dissection brains were kept on ice until thawed. For the dissection each individual brain was placed on an ice-cold iron platform. Each brain hemisphere was dissected into Cortex (all cortical areas were pull together), Hippocampus, Striatum, Thalamic area (Thalamus and hypothalamus), Mid Brain, Brain Stem and Cerebellum, put in individual 1.5 mL Eppendorf tubes, frozen on Dry Ice and stored at −80° C.

RNA Extraction and Quantification from Murine Brain Tissue

Only the cortex, hippocampus and striatum were used for total RNA extraction using Trizol (ThermoFisher Scientific; Cat. No. 15596018) following manufacturer protocol. For tissue disaggregation we used a 2 mL Potter-Elvehjem tissue grinder (DKW Life Sciences; Cat. No. 358003). To avoid RNA cross contamination the PTFE pestle was cleaned after each use with 1% SDS in DEPC-treated milliQ H2O, 100% Ethanol and finally DEPC-treated milliQ H2O. After precipitation, the RNA samples were air-dried and resuspended in 50 μL of DEPC-treated milliQ H2O. Concentrations were determined using 1 μL of the sample in a Nanodrop2000 spectrophotometer according to manufacturer's instructions.

### TrkB Activity Reporter Assay

In order to determine BDNF-specific activity in ExoBDNF preparations, a commercially-available reporter assay was used according to the manufacturer's instructions (PathHunter TrkB functional assay kit, Eurofins). Briefly, U2OS cells expressing the BDNF receptor TrkB linked to a luminescent reporter construct were plated according the manufacturer's instructions. Subsequently, these U2OS cell cultures were treated with either recombinant human BDNF, vehicle control, ExoPr0 or ExoBDNF. A substrate that generates luminescent signals in the presence of activated reporter construct was then added to the cultures and luminescence was detected using a SpectraMax i3x plate reader (Molecular Devices).

### Evaluation of Survival of Rat Retinal Ganglion Cells

In order to determine the pro-survival effect of ExoBDNF on rat retinal ganglion cells (RGC), bulk preparations of retinal cells (containing RGC) were harvested from healthy rats and seeded into 8-well chamber slides according to the method described in Mead & Tomarev, Stem Cells Translational Medicine, Vol 6, Issue 4, April 2017, p 1273-1285. Retinal cell cultures were subsequently treated for 72 hr with either ExoBDNF, ExoPro or vehicle control (Veh). Following treatment, cultures were fixed and stained for beta-Ill tubulin expression (a marker of RGCs), and the proportion of RGCs showing evidence of neurite outgrowth was determined by immunofluorescent microscopy.

### Results

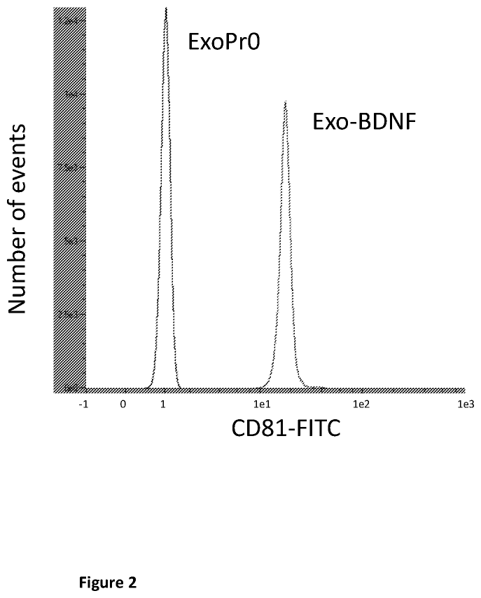

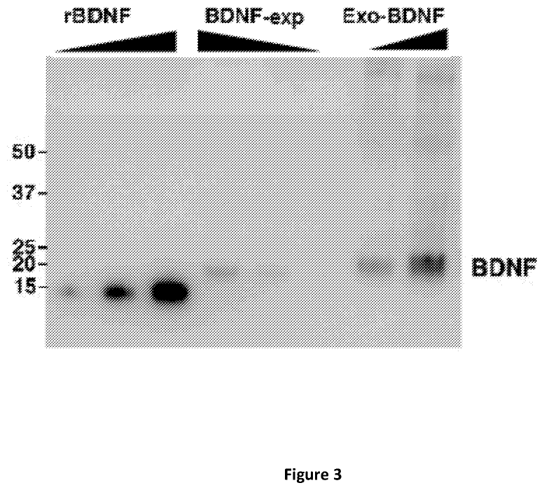

The Results are shown in FIGS. 2 to 5, 9 and 10

Firstly, ExoBDNF exosomes bind to the antigen-binding domain of TrkB as shown in FIG. 2. This histogram represents flow cytometric analysis of beads carrying Trk-B antigen binding domain after incubation with either native exosomes (ExoPr0) or ExoBDNF. Following incubation, beads were washed and incubated with anti-CD81-FITC antibodies and the fluorescence was measured by flow cytometry. Native exosomes=black overlay, ExoBDNF=red overlay. This demonstrates that the BDNF molecule expressed on the exosomes presented in a way that is accessible to TrkB receptors and is properly folded and dimerised which enables a specific interaction with the antigen-binding domain.

The results in FIG. 3 demonstrate that BDNF is detected in ExoBDNF preparations by Western Blot. Commercially available recombinant purified BDNF protein (rBDNF), ectopically expressed BDNF isolated from HEK293T cells or ExoBDNF were solubilised and analysed by Western blot as described above. Lysates were probed for BDNF content using a BDNF-specific primary antibody. These data again confirm that the BDNF expressed in ExoBDNF is structurally similar to that of native BDNF. It also enables quantification of the BDNF incorporated into the exosomes, using a standard curve formed from known quantities of rBDNF.

As shown in FIG. 4, ExoBDNF drives neurite outgrowth in an in vitro dopaminergic neuron model. Dopaminergic neurons were derived from SH-SY5Y cells and subsequently treated with vehicle control, ExoBDNF or rBDNF for 5 days. Images of the same location within the cell culture were recorded at t=0 and t=5 days post-treatment. These data demonstrate a clear increase in neurites (filamentous processes in the image) driven by ExoBDNF treatment, suggesting the ExoBDNF treatment engages target receptors in living cells and this drives the expected phenotypic and morphological changes associated with BDNF activity, namely neurite outgrowth.

FIG. 5 demonstrates that ExoBDNF drives transcription of the BDNF target gene, c-fos, in murine brains in vivo. Murine hosts were treated with vehicle control, rBDNF (2.5 ng per dose), native exosomes or ExoBDNF (50 pg BDNF per dose) by intrathecal administration as described above. c-fos mRNA levels analyzed by qPCR from the hippocampus and striatum from each animal (N=5), using TBP and 18S as internal controls and normalized. 1 way ANOVA followed by Tukey's multiple comparison test was assayed in order to determine statistical significance. ** P<0.01. Non statistical significance was observed in the cortex between any of the treatments. This data demonstrates that the activity of the ExoBDNF is detectable in vivo, and confirms engagement of target receptors and upregulation of the expected target gene, c-fos. It also highlights the advantage of ExoBDNF over recombinant BDNF when administered in vivo, since the amount of rBDNF administered was 50-fold higher than that delivered ExoBDNF.

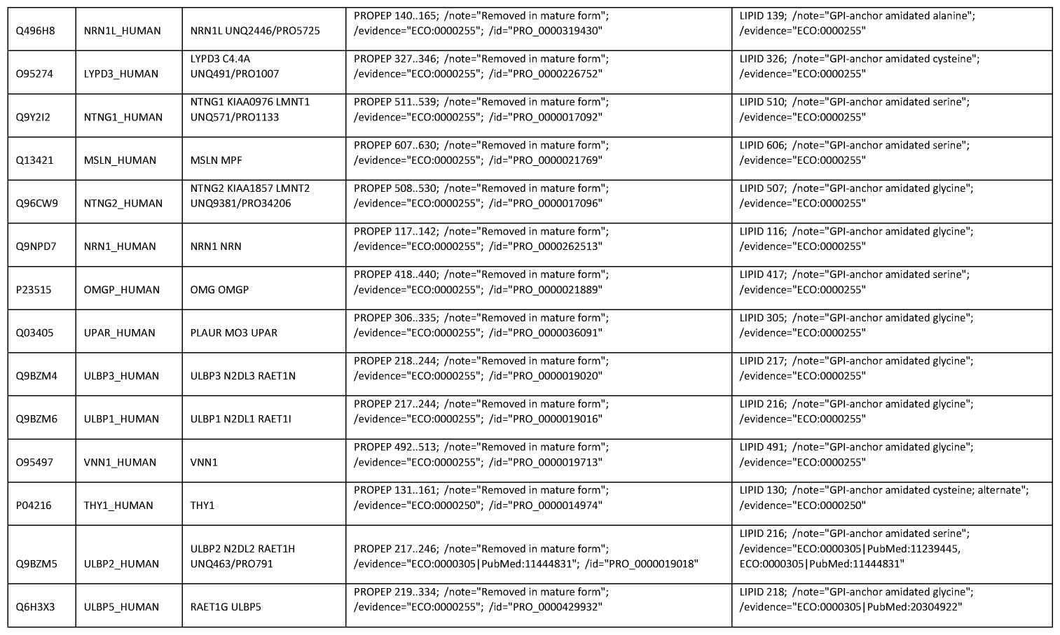

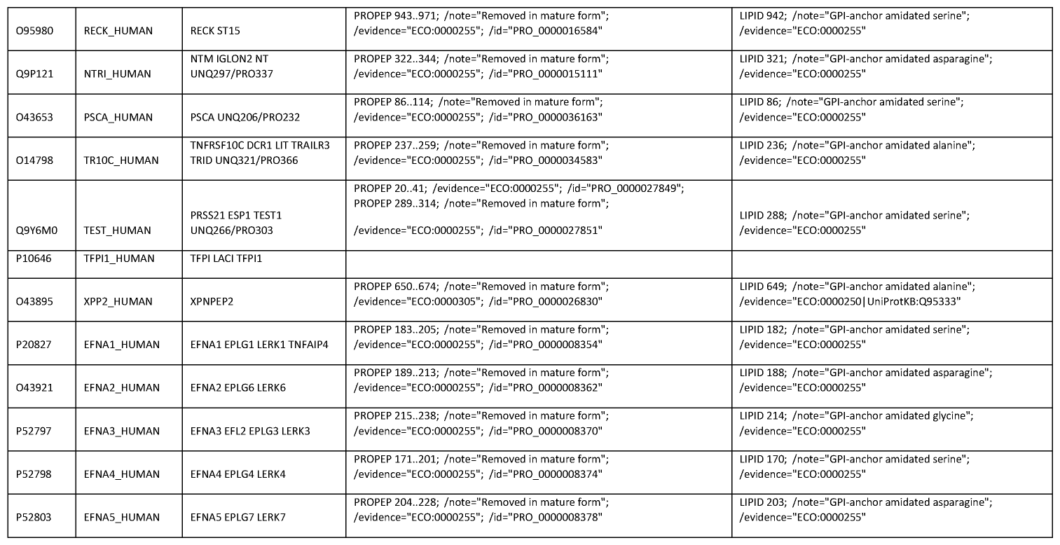

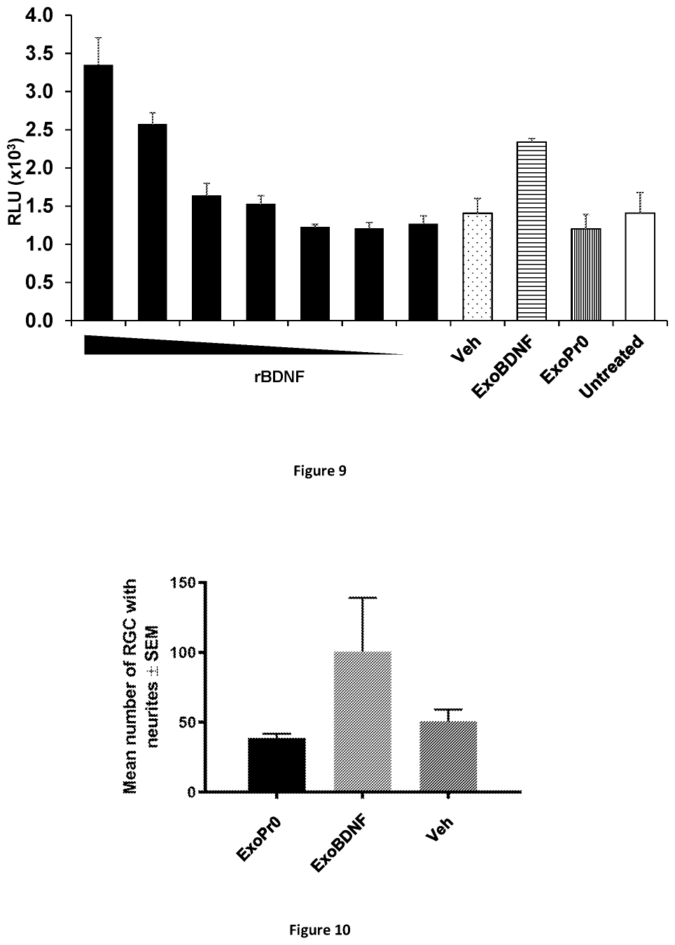

FIG. 9 evaluates BDNF-specific activity in ExoBDNF using a TrkB activity reporter assay. The bar chart represents BDNF shows activity detected using a luminescent TrkB activity reporter assay described in the methods. Black bars=dilution series of recombinant human BDNF. Pattern filled bars represent vehicle control (Veh), ExoBDNF, native exosomes (ExoPr0) and untreated wells respectively. Error bars show standard deviation calculated using technical replicates. These data confirm ExoBDNF activates TrkB signalling suggesting activity which is not present in unmodified exosomes.

FIG. 10 compares survival of neurite-bearing RGCs. Retinal cell preparations were obtained and seeded into culture as described in the methods. Bar chart represents the number of surviving RGCs detected with neurites after treatment for 72 hr with either ExoPr0, ExoBDNF or vehicle control (Veh). Error bars show standard error of the mean (SEM) calculated using technical replicates. These data suggest ExoBDNF confers an advantage in survival of neurite-bearing RGC after mechanical insult.

### Exemplary Sequences

pLVX-BDNF-Val-T2A-BSD Confirmed Sequence DNA

Nucleotide:
(SEQ ID NO: 20)
ATGACCATCCTTTTCCTTACTATGGTTATTTCATACTTTGGTTGCATGAA
GGCTGCCCCCATGAAAGAAGCAAACATCCGAGGACAAGGTGGCTTGGCCT
ACCCAGGTGTGCGGACCCATGGGACTCTGGAGAGCGTGAATGGGCCCAAG
GCAGGTTCAAGAGGCTTGACATCATTGGCTGACACTTTCGAACACGTGAT
AGAAGAGCTGTTGGATGAGGACCAGAAAGTTCGGCCCAATGAAGAAAACA
ATAAGGACGCAGACTTGTACACGTCCAGGGTGATGCTCAGTAGTCAAGTG
CCTTTGGAGCCTCCTCTTCTCTTTCTGCTGGAGGAATACAAAAATTACCT
AGATGCTGCAAACATGTCCATGAGGGTCCGGCGCCACTCTGACCCTGCCC
GCCGAGGGGAGCTGAGCGTGTGTGACAGTATTAGTGAGTGGGTAACGGCG
GCAGACAAAAAGACTGCAGTGGACATGTCGGGCGGGACGGTCACAGTCCT
TGAAAAGGTCCCTGTATCAAAAGGCCAACTGAAGCAATACTTCTACGAGA
CCAAGTGCAATCCCATGGGTTACACAAAAGAAGGCTGCAGGGGCATAGAC
AAAAGGCATTGGAACTCCCAGTGCCGAACTACCCAGTCGTACGTGCGGGC
CCTTACCATGGATAGCAAAAAGAGAATTGGCTGGCGATTCATAAGGATAG
ACACTTCTTGTGTATGTACATTGACCATTAAATACCCATACGACGTACCA
GATTACGCTGGCGCGCCACTGGAAAATGGCGGAACCTCTCTGTCTGAAAA
AACTGTGCTGCTGCTGGTGACTCCCTTTCTGGCCGCTGCTTGGTCCCTCC
ACCCA

Amino acid:
(SEQ ID NO: 21)
Met Thr Ile Leu Phe Leu Thr Met Val Ile Ser Tyr Phe Gly Cys Met Lys Ala Ala Pro Met Lys Glu Ala Asn Ile
Arg Gly Gln Gly Gly Leu Ala Tyr Pro Gly Val Arg Thr His Gly Thr Leu Glu Ser Val Asn Gly Pro Lys Ala Gly
Ser Arg Gly Leu Thr Ser Leu Ala Asp Thr Phe Glu His Val lle Glu Glu Leu Leu Asp Glu Asp Gln Lys Val Arg
Pro Asn Glu Glu Asn Asn Lys Asp Ala Asp Leu Tyr Thr Ser Arg Val Met Leu Ser Ser Gln Val Pro Leu Glu
Pro Pro Leu Leu Phe Leu Leu Glu Glu Tyr Lys Asn Tyr Leu Asp Ala Ala Asn Met Ser Met Arg Val Arg Arg
His Ser Asp Pro Ala Arg Arg Gly Glu Leu Ser Val Cys Asp Ser Ile Ser Glu Trp Val Thr Ala Ala Asp Lys Lys
Thr Ala Val Asp Met Ser Gly Gly Thr Val Thr Val Leu Glu Lys Val Pro Val Ser Lys Gly Gln Leu Lys Gln Tyr
Phe Tyr Glu Thr Lys Cys Asn Pro Met Gly Tyr Thr Lys Glu Gly Cys Arg Gly lle Asp Lys Arg His Trp Asn Ser
Gln Cys Arg Thr Thr Gln Ser Tyr Val Arg Ala Leu Thr Met Asp Ser Lys Lys Arg Ile Gly Trp Arg Phe lle Arg
Ile Asp Thr Ser Cys Val Cys Thr Leu Thr Ile Lys Tyr Pro Tyr Asp Val Pro Asp Tyr Ala Gly Ala Pro Leu Glu
Asn Gly Gly Thr Ser Leu Ser Glu Lys Thr Val Leu Leu Leu Val Thr Pro Phe Leu Ala Ala Ala Trp Ser Leu His
Pro

### Or

(SEQ ID NO: 22)
MTILFLTMVISYFGCMKAAPMKEANIRGQGGLAYPGVRTHGTLESVNGPK
AGSRGLTSLADTFEHVIEELLDEDQKVRPNEENNKDADLYTSRVMLSSQV
PLEPPLLFLLEEYKNYLDAANMSMRVRRHSDPARRGELSVCDSISEWVTA
ADKKTAVDMSGGTVTVLEKVPVSKGQLKQYFYETKCNPMGYTKEGCRGID
KRHWNSQCRTTQSYVRALTMDSKKRIGWRFIRIDTSCVCTLTIKYPYDVP
DYAGAPLENGGTSLSEKTVLLLVTPFLAAAWSLH

BDNF shown in plain text

### HA Purification Tag Shown in Bold

### Linker Shown Underlined

GPI-CD50 shown in Italics

For the avoidance of doubt, the amino acid sequences of the BDNF, HA purification tag, linker and GPI-CD59 components of the exemplified construct are shown in the table below:

BDNF (mature sequence underlined)
(SEQ ID NO: 23)
MTILFLTMVISYFGCMKAAPMKEANIRGQGGLAYPGVRTHGTLE
SVNGPKAGSRGLTSLADTFEHVIEELLDEDQKVRPNEENNKDAD
LYTSRVMLSSQVPLEPPLLFLLEEYKNYLDAANMSMRVRRHSDP
ARRGELSVCDSISEWVTAADKKTAVDMSGGTVTVLEKVPVSKG
QLKQYFYETKCNPMGYTKEGCRGIDKRHWNSQCRTTQSYVRAL
TMDSKKRIGWRFIRIDTSCVCTLTIK
HA Purification Tag
(SEQ ID NO: 3)
YPYDVPDYA
Linker
(SEQ ID NO: 2)
GAPLE
GPI-CD59
(SEQ ID NO: 1)
NGGTSLSEKTVLLLVTPFLAAAWSLH

1. A fusion protein comprising a protein of interest and a carboxy-terminal glycosylphosphatidylinositol (GPI) anchor peptide sequence, optionally wherein the protein of interest and GPI anchor protein are separated by a linker peptide.

2. A fusion protein according to claim 1, wherein the linker peptide is present and comprises the sequence GAPLE (SEQ ID NO: 2) or a functional variant thereof.

3. A fusion protein according to claim 1 or claim 2, wherein the GPI anchor protein comprises the GPI anchoring region from human CD59, optionally having the sequence NGGTSLSEKTVLLLVTPFLAAAWSLH (SEQ ID NO: 1) or a functional variant thereof having at least 70% identity to that sequence.

4. A fusion protein comprising a protein of interest, a linker peptide and a final carboxy-terminal residue, wherein the final carboxy-terminal residue is covalently linked to glycosylphosphatidylinositol (GPI).

5. A fusion protein according to claim 4, wherein:
the linker peptide comprises or consists of the sequence GAPLE peptide or a functional variant thereof; and/or
the final carboxy-terminal residue is asparagine, alanine, aspartic acid, cysteine, glycine, or serine; and/or
the GPI is embedded in a biological lipid membrane.

6. A fusion protein according to claim 5, wherein the biological lipid membrane is an extracellular vesicle membrane, optionally an exosome membrane.

7. A fusion protein according to any preceding claim, wherein the protein of interest is:
a therapeutic protein, a ligand, a receptor, a growth factor, cytokine, an antibody or an antigen-binding fragment thereof, or a T cell receptor or antigen-binding fragment thereof; or
a neurotrophin.

8. A fusion protein according to any preceding claim, wherein the protein of interest is Brain-Derived Neurotrophic Factor (BDNF).

9. A fusion protein according to any preceding claim, wherein the protein of interest comprises a mature sequence and optionally a pro-peptide and/or signal sequence.

10. A fusion protein according to any preceding claim, in the form of a biologically-active homodimer.

11. A fusion protein according to any preceding claim, comprising a purification tag, optionally wherein the purification tag comprises or consists of the sequence YPYDVPDYA or a functional variant thereof.

12. A fusion protein according to any preceding claim, wherein:
the fusion protein is according to any of claims 1 to 3 or 7 to 11 and the order of components N-terminus to C-terminus is: protein of interest-optional purification tag-linker peptide-GPI anchor protein; or
the fusion protein is according to any of claims 4 to 11 and the order of components N-terminus to C-terminus is: protein of interest-optional purification tag-linker peptide-GPI-optional EV membrane.

13. A fusion protein according to claim 12, wherein the protein of interest comprises or consists of mature BDNF, the optional purification tag comprises or consists of YPYDVPDYA (SEQ ID NO: 3), the linker peptide comprises or consists of GAPLE (SEQ ID NO: 2), and the GPI-anchor protein is NGGTSLSEKTVLLLVTPFLAAAWSLH (SEQ ID NO: 1).

14. A fusion protein according to any preceding claim, comprising:
at least 80% or at least 90% identity to the sequence MTILFLTMVISYFGCMKAAPMKEANIRGQGGLAYPGVRTHGTLESVNGPKAGSRGLTS LADTFEHVIEELLDEDQKVRPNEENNKDADLYTSRVMLSSQVPLEPPLLFLLEEYKNYL DAANMSMRVRRHSDPARRGELSVCDSISEWVTAADKKTAVDMSGGTVTVLEKVPVSK GQLKQYFYETKCNPMGYTKEGCRGIDKRHWNSQCRTTQSYVRALTMDSKKRIGWRFI RIDTSCVCTLTIKGAPLENGGTSLSEKTVLLLVTPFLAAAWSLH (SEQ ID NO: 4); or
at least 80% or at least 90% identity to the sequence SDPARRGELSVCDSISEWVTAADKKTAVDMSGGTVTVLEKVPVSKGQLKQYFYETKC NPMGYTKEGCRGIDKRHWNSQCRTTQSYVRALTMDSKKRIGWRFIRIDTSCVCTLTIK GAPLEN (SEQ ID NO:24).

15. A polynucleotide encoding a fusion protein according to any preceding claim.

16. A plasmid, virus or vector comprising the polynucleotide of claim 15.

17. A plasmid, virus or vector according to claim 16, wherein the polynucleotide encoding a fusion protein is under the control of a promoter sequence, optionally an inducible promoter.

18. An extracellular vesicle comprising a fusion protein according to any of claims 4 to 14, wherein the GPI is anchored in the extracellular vesicle membrane.

19. An extracellular vesicle according to claim 18, wherein the fusion protein is present as a homodimer in the extracellular vesicle membrane.

20. An extracellular vesicle according to claim 18 or claim 19, comprising an additional exogenous protein, nucleic acid, lipid, drug, prodrug, therapeutic agent or diagnostic agent.

21. A host cell comprising a fusion protein according to any of claims 1 to 14, a polynucleotide according to claim 15, a plasmid, virus or vector according to claim 16 or claim 17, or an exosome according to any of claims 18 to 20.

22. A pharmaceutical composition comprising a fusion protein of any of claims 1 to 15, a polynucleotide according to claim 15, a plasmid, virus or vector according to claim 16 or claim 17, an extracellular vesicle according to any of claims 18 to 20, or a host cell according to claim 21, and a pharmaceutically acceptable carrier, diluent or excipient.

23. A fusion protein according to any of claims 1 to 14, a polynucleotide according to claim 15 or a plasmid, virus or vector according to claim 16 or claim 17, or an exosome according to any of claims 18 to 20, or a cell according to claim 21, for use in a method of treatment or a method of diagnosis.

24. A fusion protein for use, polynucleotide for use, plasmid, virus or vector for use, or exosome for use according to claim 23, wherein the treatment is the treatment or prevention of a central nervous system disease or disorder, or prevention or treatment of a disease or disorder of the eye.

25. A method of expressing a fusion protein according to any of claims 1 to 14 or of producing an exosome according to any of claims 18 to 20, wherein the method comprises expressing the fusion protein in a host cell, optionally from a recombinant expression cassette or expression vector,
optionally wherein the host cell is a conditionally-immortalised cell line, optionally the CTX0E03 cell line.

26. A method according to claim 25, comprising the step of harvesting or collecting exosomes comprising the fusion protein from the host cell or host cell media.

27. A method of expressing a fusion protein according to claim 25 or claim 26, wherein the expression comprises:
cleavage of a pro-peptide sequence present on the protein of interest; and/or
cleavage of C-terminal residues of the GPI anchor protein sequence and attachment of GPI to the remaining residues of the GPI anchor protein sequence; and/or
embedding the fusion protein into the exosome membrane via GPI attached to the GPI anchor sequence.

28. A method of expressing a fusion protein according to claim 27, wherein the cleavage(s) is carried out co-translationally or post-translationally, by endogenous enzymes in the host cell.


SEQUENCE LISTING (24 sequences)

SEQ ID NO: 1 - protein, 26 aa, Homo sapiens
NGGTSLSEKTVLLLVTPFLAAAWSLH

SEQ ID NO: 2 - protein, 5 aa, synthetic construct
GAPLE

SEQ ID NO: 3 - protein, 9 aa, synthetic construct
YPYDVPDYA

SEQ ID NO: 4 - protein, 275 aa, synthetic construct
MTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKGAPLENGGTSLSEKTVLLLVTPFLAAAWSLH

SEQ ID NO: 5 - protein, 122 aa, synthetic construct
HSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKGAPLEN

SEQ ID NO: 6 - protein, 247 aa, Homo sapiens
MTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 7 - protein, 25 aa, Homo sapiens
GGTSLSEKTVLLLVTPFLAAAWSLH

SEQ ID NO: 8 - protein, 26 aa, Homo sapiens
SASSNISGGIFLFFVANAIIHLFCFS

SEQ ID NO: 9 - protein, 29 aa, Homo sapiens
SGTTRLLSGHTCFTLTGLLGTLVTMGLLT

SEQ ID NO: 10 - protein, 31 aa, Homo sapiens
GAAPQPGPAHLSLTITLLMTARLWGGTLLWT

SEQ ID NO: 11 - protein, 5 aa, synthetic construct
AGELP

SEQ ID NO: 12 - protein, 5 aa, synthetic construct
AGLEP

SEQ ID NO: 13 - protein, 5 aa, Homo sapiens
EGLAP

SEQ ID NO: 14 - protein, 5 aa, synthetic construct
ELPAG

SEQ ID NO: 15 - protein, 5 aa, synthetic construct
GGPGE

SEQ ID NO: 16 - protein, 5 aa, synthetic construct
AAPAE

SEQ ID NO: 17 - protein, 5 aa, synthetic construct
VVPVE

SEQ ID NO: 18 - protein, 5 aa, synthetic construct
LLPLE

SEQ ID NO: 19 - protein, 5 aa, synthetic construct
IIPIE

SEQ ID NO: 20 - DNA, 855 bp, synthetic construct
  source 1..855: mol_type = other DNA
atgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacgtgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaatacccatacgacgtaccagattacgctggcgcgccactggaaaatggcggaacctctctgtctgaaaaaactgtgctgctgctggtgactccctttctggccgctgcttggtccctccaccca

SEQ ID NO: 21 - protein, 285 aa, synthetic construct
MTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKYPYDVPDYAGAPLENGGTSLSEKTVLLLVTPFLAAAWSLHP

SEQ ID NO: 22 - protein, 284 aa, synthetic construct
MTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKYPYDVPDYAGAPLENGGTSLSEKTVLLLVTPFLAAAWSLH

SEQ ID NO: 23 - protein, 244 aa, Homo sapiens
MTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRRHSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIK

SEQ ID NO: 24 - protein, 121 aa, synthetic construct
SDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKGAPLEN